(19) United States
(12) Patent Application Publication
(10) Pub. No.: US 2025/0161754 A1
(43) Pub. Date: May 22, 2025

(54) EXERCISE COUNTING METHOD AND ELECTRONIC DEVICE SUPPORTING SAME

(71) Applicant: SAMSUNG ELECTRONICS CO., LTD., Suwon-si (KR)

(72) Inventors: Hyeonseong KIM, Suwon-si (KR); Chanung PARK, Suwon-si (KR); Jeongmin PARK, Suwon-si (KR)

(73) Assignee: SAMSUNG ELECTRONICS CO., LTD., Suwon-si (KR)

(21) Appl. No.: 19/028,505

(22) Filed: Jan. 17, 2025

Related U.S. Application Data
(63) Continuation of application No. PCT/KR2023/009058, filed on Jun. 28, 2023.

(30) Foreign Application Priority Data
Jul. 29, 2022 (KR) 10-2022-0095055
Sep. 1, 2022 (KR) 10-2022-0110657

(51) Int. Cl. A63B 24/00; A63B 71/06

(52) U.S. Cl. CPC A63B 24/0075; A63B 71/0622; A63B 2024/0068; A63B 2220/17; A63B 2220/803; A63B 2230/62

(57) ABSTRACT

An electronic device includes: a display; at least one sensor; memory storing instructions; and at least one processor operatively connected to the display, the at least one sensor, and the memory and configured to execute the instructions to: provide exercise guidance based on detecting an exercise start trigger, recognize an exercise-ready posture of a user for a specified time period based on sensor data from at least one specified sensor of the at least one sensor, provide exercise posture information related to a type of an exercise corresponding to the exercise-ready posture, drive a recognition schema for counting the exercise corresponding to the exercise-ready posture, perform exercise counting based on the recognition schema, and provide exercise information based on the exercise counting.

### CROSS-REFERENCE TO RELATED APPLICATION(S)

This application is a continuation of International Application No. PCT/KR2023/009058, filed on Jun. 28, 2023, which is based on and claims priority to Korean Patent Applications No. 10-2022-0095055, filed on Jul. 29, 2022 and No. 10-2022-0110657, filed on Sep. 1, 2022, in the Korean Intellectual Property Office, the disclosures of which are incorporated by reference herein in their entireties.

### BACKGROUND

### 1. Field

This disclosure relates to a method of enhancing performance of exercise counting recognition and an electronic device that supports the same.

### 2. Description of Related Art

With the advancement of digital technology, various types of electronic devices, such as smart phones and/or wearable devices, are being widely used. Hardware components and/or software components of the electronic device are consistently developed to support and improve the function of the electronic device.

Interest in a user's health care using electronic devices has recently increased. For example, a user may perform exercise while carrying an electronic device. The electronic device may provide exercise counting for the user's exercise and related exercise information based on the exercise counting to the user. Therefore, various studies are being conducted in electronic devices to provide highly accurate exercise information according to the user's health care.

### SUMMARY

Provided are a method of enhancing the recognition performance of exercise counting in various exercise postures of a user, and an electronic device supporting the same.

Additional aspects will be set forth in part in the description which follows and, in part, will be apparent from the description, or may be learned by practice of the presented embodiments.

According to an aspect of the disclosure, an electronic device may include: a display; at least one sensor; a memory storing instructions; and at least one processor operatively connected to the display, the at least one sensor, and the memory, wherein the at least one processor is configured to execute the instructions to: provide exercise guidance based on detecting an exercise start trigger, recognize an exercise-ready posture of a user for a specified time period based on sensor data from at least one specified sensor of the at least one sensor, provide exercise posture information related to a type of an exercise corresponding to the exercise-ready posture, drive a recognition schema for counting the exercise corresponding to the exercise-ready posture, perform exercise counting based on the recognition schema, and provide exercise information based on the exercise counting.

The at least one processor may be further configured to execute the instructions to recognize the exercise counting based on a countable signal from the at least one specified sensor.

The at least one processor may be further configured to execute the instructions to: recognize an exercise posture based on the recognized exercise-ready posture, and provide, during the recognition of the exercise posture, exercise posture information related to the recognized exercise-ready posture by displaying at least one of visual images or text on the display.

The at least one processor may be further configured to execute the instructions to: compare the exercise posture with a specified reference exercise posture corresponding to the recognized exercise-ready posture, and determine whether the exercise posture is being maintained based on a similarity between the exercise posture and the specified reference exercise posture.

The at least one processor may be further configured to execute the instructions to: correct the exercise-ready posture in a state in which the exercise posture does not correspond to the specified reference exercise posture, determine a first recognition schema as the recognition schema for counting the exercise corresponding to the corrected exercise-ready posture, based on the exercise-ready posture having been corrected, determine a second recognition schema, different from the first recognition schema, as the recognition schema for counting the exercise corresponding to the exercise-ready posture in a state in which the exercise posture corresponds to the specified reference exercise posture, and continuously accumulate and provide the exercise information according to the counting of the exercise based on the first recognition schema or the second recognition schema.

The at least one processor may be further configured to execute the instructions to: determine whether the exercise-ready posture is maintained for a predetermined time period based on a result of recognizing the exercise-ready posture during the specified time period, and drive the recognition schema corresponding to the exercise-ready posture in a state in which the exercise-ready posture is maintained for the predetermined time period.

The at least one processor may be further configured to execute the instructions to: detect an exercise counting candidate section based on the sensor data from the at least one specified sensor, filter an exercise motion and a non-exercise motion for the exercise counting candidate section, calculate an exercise posture at a start point and an end point of the exercise counting candidate section, and calculate a similarity between the exercise-ready posture and the exercise posture of the exercise counting candidate section.

The at least one processor may be further configured to execute the instructions to: extract a countable signal through signal processing of the sensor data from the at least one specified sensor, and detect the exercise counting candidate section based on the extracted countable signal.

The at least one processor may be further configured to execute the instructions to: detect zero crossing points for the extracted countable signal, detect peaks and valleys within a zero crossing section, determine whether the peaks and valleys satisfy a specified condition based on whether the peaks and valleys pass through a predefined upper boundary and lower boundary, check a sequence in which the countable signal passes through the predefined upper boundary and lower boundary within the zero crossing section in a state in which the specified condition is satisfied, and determine a zero crossing section of the countable signal that satisfies the specified condition as the exercise counting candidate section.

The at least one processor may be further configured to execute the instructions to perform filtering of exercise counting false recognition based on characteristic parameters for distinguishing between the exercise motion and the non-exercise motion, where the characteristic parameters include at least one of an amount of change in acceleration, an acceleration peak-valley interval, an amount of change in angular velocity, or an amount of change in atmospheric pressure.

The at least one processor may be further configured to execute the instructions to: determine that the user is maintaining the exercise posture in a state in which the similarity exceeds a threshold, and update the exercise information.

The at least one processor may be further configured to execute the instructions to: determine that the exercise posture is not being maintained in a state in which the similarity is equal to or less than a threshold, and correct the exercise-ready posture.

The at least one processor may be further configured to execute the instructions to: accumulate the exercise counting candidate section in a state in which the similarity is equal to or less than the threshold, determine whether there is a correction history for the exercise-ready posture in a state in which a number of candidate sections exceeds a predetermined count, correct the exercise-ready posture in a state in which there is no correction history for the exercise-ready posture, and provide guidance on the exercise posture in a state in which there is the correction history for the exercise-ready posture.

The electronic device may further include communication circuitry, and the at least one processor may be further configured to execute the instructions to: control the communication circuitry to establish wireless communication with an external device based on detecting the exercise start trigger, transmit visual information related to exercise coaching to the external device, and enable the visual information related to the exercise coaching to be displayed through at least one of the display of the electronic device or a display of the external device.

According to an aspect of the disclosure, a method of operating an electronic device may include: providing exercise guidance based on detecting an exercise start trigger; recognizing an exercise-ready posture of a user for a specified time period based on sensor data from at least one specified sensor of the electronic device; providing exercise posture information related to a type of an exercise corresponding to the exercise-ready posture; driving a recognition schema for counting the exercise corresponding to the exercise-ready posture; performing exercise counting based on the recognition schema; and providing exercise information based on the exercise counting.

According to an aspect of the disclosure, a non-transitory computer-readable recording medium storing computer-executable instructions that, when executed by a processor of an electronic device individually and/or collectively, cause the electronic device to perform operations is provided. The operations may include based on detecting an exercise start trigger, providing exercise guidance, recognizing an exercise-ready posture of a user for a specified time period based on sensor data from at least one specified sensor of the electronic device, providing exercise posture information related to a type of an exercise corresponding to the exercise-ready posture, driving a recognition schema for counting the exercise corresponding to the exercise-ready posture, performing exercise counting based on the recognition schema, and providing exercise information based on the exercise counting.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram illustrating an example electronic device in a network environment according to various embodiments;

FIG. 2 is a view schematically illustrating a configuration of an electronic device according to an embodiment of the present disclosure;

FIG. 3 is a flowchart illustrating a method of operating an electronic device according to an embodiment of the present disclosure;

FIG. 4 is a view illustrating an example of a user interface supporting exercise coaching in an electronic device according to an embodiment of the present disclosure;

FIG. 5 is a flowchart illustrating a method of operating an electronic device according to an embodiment of the present disclosure;

FIG. 6 is a view illustrating an example of a user interface supporting exercise coaching in an electronic device according to an embodiment of the present disclosure;

FIG. 7 is a flowchart illustrating a method of operating an electronic device according to an embodiment of the present disclosure;

FIG. 8 is a view illustrating an example of a user interface supporting exercise coaching in an electronic device according to an embodiment of the present disclosure;

FIG. 9 is a flowchart illustrating a method of operating an electronic device according to an embodiment of the present disclosure;

FIG. 10 is a flowchart illustrating a method of operating an electronic device according to an embodiment of the present disclosure;

FIG. 11 is a reference view for describing exercise counting candidate section detection according to an embodiment of the present disclosure;

FIG. 12A and FIG. 12B are reference views for describing exercise counting candidate section detection according to an embodiment of the present disclosure;

FIG. 13A and FIG. 13B are reference views for describing exercise counting candidate section detection according to an embodiment of the present disclosure;

FIG. 14 is a flowchart illustrating a method of operating an electronic device according to an embodiment of the present disclosure;

FIG. 15 is a reference view for describing a false recognition filtering operation of exercise counting according to an embodiment of the present disclosure;

FIG. 16 is a reference view for describing a false recognition filtering operation of exercise counting according to an embodiment of the present disclosure;

FIG. 17 is a flowchart illustrating a method of operating an electronic device according to an embodiment of the present disclosure;

FIG. 18 is a reference view for describing the determination of whether an exercise posture is being maintained, according to an embodiment of the present disclosure; and

FIG. 19 is a flowchart illustrating a method of operating an electronic device according to an embodiment of the present disclosure.

### DETAILED DESCRIPTION

Hereinafter, example embodiments of the disclosure will be described in detail with reference to the accompanying drawings. The same reference numerals are used for the same components in the drawings, and redundant descriptions thereof will be omitted. The embodiments described herein are example embodiments, and thus, the disclosure is not limited thereto and may be realized in various other forms. It is to be understood that singular forms include plural referents unless the context clearly dictates otherwise. The terms including technical or scientific terms used in the disclosure may have the same meanings as generally understood by those skilled in the art.

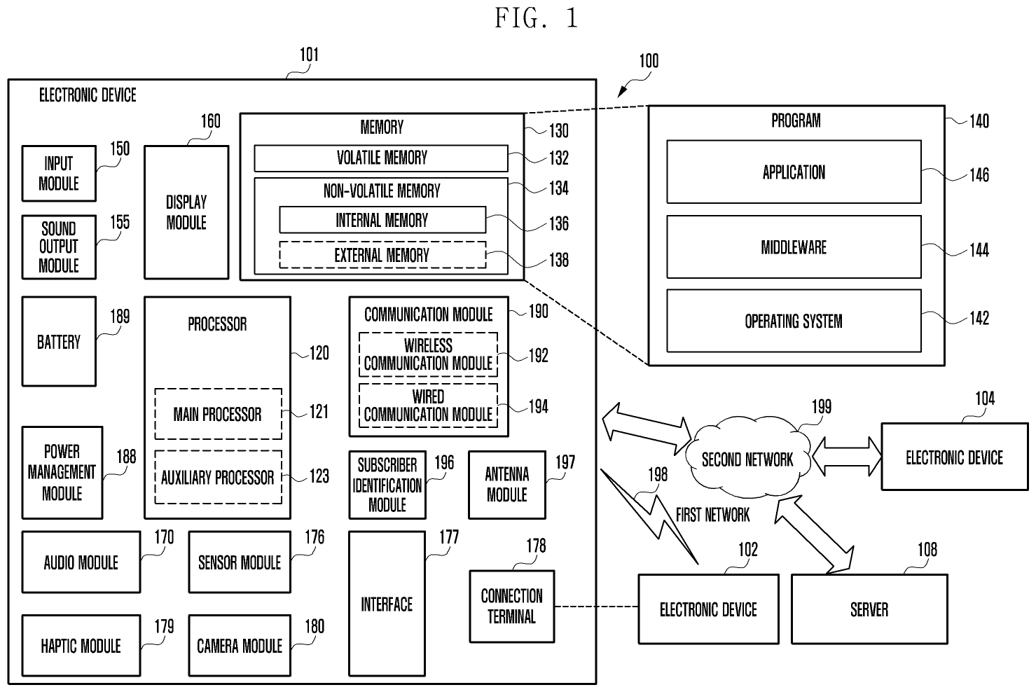

FIG. 1 is a block diagram illustrating an example electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In various embodiments, at least one of the components (e.g., the connecting terminal 178) may be omitted from the electronic device 101, or one or more other components may be added in the electronic device 101. In various embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to an embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to an embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element including a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (QEC), or client-server computing technology may be used, for example. The electronic device 101 may provide ultra low-latency services using, e.g., distributed computing or mobile edge computing. In an embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

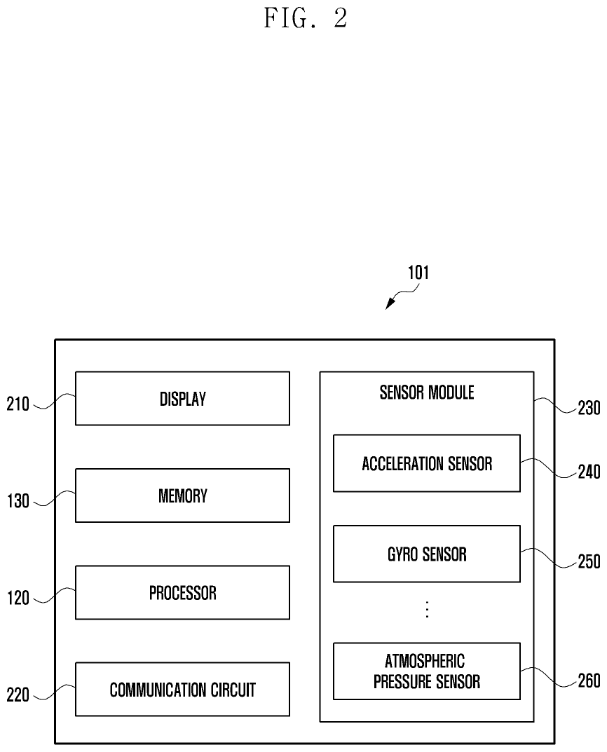

FIG. 2 is a view schematically illustrating a configuration of an electronic device according to an embodiment of the present disclosure.

An electronic device 101 according to an embodiment of the present disclosure may include various devices that is able to count a user's exercise while being worn on a user's body (e.g., arm) or held in the user's hand. According to an embodiment, the electronic device 101 may include a mobile terminal that is wearable on a user's body in cooperation with a wearable device and an apparatus that is wearable (or attachable) to the user's body (e.g., arm). According to an embodiment, the wearable device may include various forms of devices, such as a watch type, ring type, and/or band type. According to an embodiment, the mobile terminal may include a smart phone, a camera, and a multimedia player. In an embodiment of the present disclosure, for convenience of description, the electronic device 101 is described as a wearable device (e.g., a watch) by way of example. However, various embodiments of the present disclosure are not limited to wearable devices.

With reference to FIG. 2, the electronic device 101 according to an embodiment of the present disclosure may include a display 210, a memory 130, a communication circuit 220, a sensor module 230, and/or a processor 120. According to an embodiment, the electronic device 101 may not include at least one constituent element (e.g., display 210 and/or communication circuit 220). According to an embodiment, the electronic device 101 may include one or more other constituent elements (e.g., camera module 180, power management module 188, and/or battery 189 in FIG. 1). For example, the electronic device 101 may include all or at least some of the constituent elements of the electronic device 101 as described in the description with reference to FIG. 1.

According to an embodiment, the display 210 may correspond to the display module 160 as described in the description with reference to FIG. 1. According to an embodiment, the display 210 may visually provide various information to the exterior (e.g., user) of the electronic device 101. According to an embodiment, the display 210 may visually provide various information related to exercise coaching under the control of the processor 120.

According to an embodiment, the display 210 may include a touch detection circuit (or touch sensor), a pressure sensor capable of measuring the intensity of touch, and/or a touch panel (e.g., digitizer) that detects a magnetic field-type stylus pen. According to an embodiment, the display 210 may detect a touch input and/or a hovering input (or proximity input) by measuring changes in signals (e.g., voltage, light quantity, resistance, electromagnetic signal, and/or charge amount) at specific positions on the display 210, on the basis of the touch detection circuit, pressure sensor, and/or touch panel. According to an embodiment, the display 210 may be configured as a liquid crystal display (LCD), an organic light-emitting diode (OLED), and an active matrix organic light-emitting diode (AMOLED). According to an embodiment, the display 210 may be configured as a flexible display.

According to an embodiment, the display 210 may include the display of an external device (e.g., TV, display device) that is connected to the electronic device 101 via wireless communication. For example, the electronic device 101 may transmit visual information related to exercise coaching to an external device connected via wireless communication, allowing the external device to display the visual information related to exercise coaching. For example, the visual information related to exercise coaching may be displayed through the display 210 of the electronic device 101 and/or the display of an external device.

According to an embodiment, the communication circuit 220 may support a legacy network (e.g., 3G network and/or 4G network), a 5G network, out-of-band (OOB) communication, and/or next-generation communication technologies (e.g., new radio (NR) technology). According to an embodiment, the communication circuit 220 may correspond to the wireless communication module 192 as illustrated in FIG. 1. According to an embodiment, the communication circuit 220 may establish wireless communication with a specified external device and transmit visual information related to exercise coaching to the specified external device.

According to an embodiment, the electronic device 101 may perform communication with an external device (e.g., server 108 in FIG. 1 and/or other electronic devices 102 and 104) through a network using the communication circuit 220. According to an embodiment, the communication circuit 220 may transmit data generated by the electronic device 101 to an external device and may receive data transmitted from the external device.

According to an embodiment, the memory 130 may correspond to the memory 130 as described in the description with reference to FIG. 1. According to an embodiment, the memory 130 may store various data used by the electronic device 101. In an embodiment, the data may include, for example, applications (e.g., program 140 in FIG. 1) and input data or output data for commands related to the applications. In an embodiment, the data may include sensor data obtained from the sensor module 230 (e.g., acceleration sensor, gyro sensor data, atmospheric pressure sensor). In an embodiment, the data may include various reference data preset in the memory 130 to improve the performance of exercise counting recognition.

According to an embodiment, the memory 130 may store instructions that, when executed, cause the processor 120 to operate. For example, the application may be stored in the memory 130 as software (e.g., program 140 in FIG. 1) and executable by the processor 120. According to an embodiment, the application may be various applications capable of providing various services (e.g., healthcare service) on the electronic device 101.

According to an embodiment, the sensor module 230 may correspond to the sensor module 176 as described in the description with reference to FIG. 1. According to an embodiment, the sensor module 230 may include various sensors such as an acceleration sensor 240, a gyro sensor 250, and/or an atmospheric pressure sensor 260. According to an embodiment, the sensor module 230 may also include an attitude sensor, which may replace the acceleration sensor 240 and/or the gyro sensor 250. According to an embodiment, the electronic device 101 may perform improvement of the performance of exercise counting recognition for the user's exercise coaching on the basis of sensor data using the acceleration sensor 240, gyro sensor 250, and/or atmospheric pressure sensor 260 of the sensor module 230.

According to an embodiment, the processor 120 may perform application layer processing functions that are required by a user of the electronic device 101. According to an embodiment, the processor 120 may provide controls and commands of functions for various blocks of the electronic device 101. According to an embodiment, the processor 120 may perform calculations or data processing related to control and/or communication of the respective constituent elements of the electronic device 101. For example, the processor 120 may include at least some of the configurations and/or functions of the processor 120 in FIG. 1. The processor 120 may be operatively connected to, for example, constituent elements of the electronic device 101. The processor 120 may load commands or data received from other constituent elements of the electronic device 101 into the memory 130, process the commands or data stored in the memory 130, and store resultant data.

According to an embodiment, the processor 120 may include processing circuitry and/or executable program elements. According to an embodiment, the processor 120 may support exercise counting in the electronic device 101 and control (or process) operations related to improving the recognition performance of exercise counting.

According to an embodiment, the processor 120 may enable exercise counting recognition for various exercise postures (e.g., squat exercise posture). According to an embodiment, the processor 120 may detect a countable signal based on specified sensors of the sensor module 230 (e.g., acceleration sensor 240, gyro sensor 250, and/or atmospheric pressure sensor 260). According to an embodiment, the processor 120 may check whether the countable signal passes through a specified predetermined section (e.g., lower boundary and upper boundary) (e.g., first information). According to an embodiment, the processor 120 may check the order (e.g., second information) in which the countable signal passes through a specified predetermined section (e.g., lower boundary and upper boundary). According to an embodiment, the processor 120 may detect an exercise counting candidate section on the basis of the first information and the second information.

According to an embodiment, the processor 120 may extract at least one characteristic parameter (e.g., amount of change in acceleration, acceleration peak-valley interval, amount of change in angular velocity, and amount of change in atmospheric pressure) for a specified sensor on the basis of sensor data from specified sensors of the sensor module 230 (e.g., acceleration sensor 240, gyro sensor 250, and/or atmospheric pressure sensor 260). According to an embodiment, the processor 120 may filter out non-exercise motions on the basis of the extracted at least one characteristic parameter. According to an embodiment, the processor 120 may achieve more accurate exercise counting recognition through filtering of non-exercise motions.

According to an embodiment, the processor 120 may allow exercise counting to be recognized even when, for example, the user's exercise posture becomes disrupted while supporting the user's exercise. According to an embodiment, the processor 120 may operate to check the exercise posture at a start point and an end point of a single exercise motion, regardless of whether the user's exercise posture is correct or disrupted. According to an embodiment, the processor 120 may check the exercise posture at the start and end points of the user's exercise motion to normally recognize the exercise count, even if the user's posture becomes disrupted during exercise.

According to an embodiment, the processor 120 may enable exercise counting recognition even when the user performs a specified exercise (e.g., squat exercise) with an exercise-ready posture different from an initial exercise-ready posture. According to an embodiment, the processor 120 may trigger a case where the exercise count is not properly recognized at the beginning of the exercise and correct the initial exercise-ready posture, thereby allowing proper recognition of the exercise count even when the user performs the exercise with an exercise posture different from the initial exercise-ready posture.

According to an embodiment, the processor 120 may interactively process operations related to exercise coaching with the electronic device 101 and an external device outside the electronic device 101 (e.g., TV, display device). According to an embodiment, the processor 120 may control the communication circuit 220 to connect with a specified external device via wireless communication upon detecting the user's exercise start (or exercise start trigger). According to an embodiment, the processor 120 may transmit visual information related to exercise coaching to an external device connected via wireless communication, allowing the external device to display the visual information related to exercise coaching. For example, the visual information related to exercise coaching may be displayed through the display 210 of the electronic device 101 and/or the display of an external device.

According to an embodiment, a detailed operation of the processor 120 of the electronic device 101 will be described with reference to the drawings described below.

According to an embodiment, the operations performed by the processor 120 may be implemented on a recording medium (or computer program product). For example, the recording medium may include a non-transitory computer-readable recording medium that records a program for executing various operations performed by the processor 120.

The embodiments disclosed herein may be implemented in a recording medium readable by a computer or similar device using software, hardware, or combinations thereof. According to hardware implementations, the operations described in an embodiment may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and/or electrical units for performing other functions.

In an embodiment, a recording medium (or computer program product) may include a computer-readable recording medium on which a program is recorded to execute operations, including: providing exercise guidance on the basis of detecting an exercise start trigger; recognizing the user's exercise-ready posture for a specified time period on the basis of sensor data from at least one specified sensor of the sensor module 230 (e.g., acceleration sensor 240, gyro sensor 250, and/or atmospheric pressure sensor 260); providing exercise posture information related to the type of exercise corresponding to the exercise-ready posture; driving a recognition schema for the user's exercise counting corresponding to the exercise-ready posture; performing exercise counting based on the recognition schema; and providing exercise information according to the exercise counting.

Hereinafter, an example of exercise counting for a squat exercise and improvement of its recognition performance in the electronic device 101 according to various embodiments will be described, but the embodiments of the present disclosure are not limited to squat exercises. For example, the various embodiments described hereinafter are provided merely to easily explain the technical content of the present disclosure and aid in understanding, using specific constituent elements (e.g., squat exercise) as examples, and are not intended to limit the scope of the present disclosure.

Therefore, the scope of the present disclosure should be interpreted that all changes or modified forms derived based on the technical spirit of the present disclosure fall within the scope of the present disclosure in addition to the embodiments disclosed herein. For example, the exercise counting and method of improving recognition performance of the exercise counting described hereinafter may be applied to improve exercise counting and its recognition performance in various exercises where the user performs the same or similar movements repeatedly using their arms, such as squat exercises (e.g., lunges, planks, push-ups, or sit-ups).

The electronic device 101 according to an embodiment of the present disclosure may include a display (e.g., display module 160 in FIG. 1 or display 210 in FIG. 2), a sensor module (e.g., sensor module 176 or 230 in FIG. 1 or FIG. 2), a memory (e.g., memory 130 in FIG. 1 or FIG. 2), and a processor (e.g., processor 120 in FIG. 1 or FIG. 2) operatively connected to the display, the sensor module, and the memory.

According to an embodiment, the processor 120 may operate to provide exercise guidance on the basis of detecting an exercise start trigger, recognize the user's exercise-ready posture for a specified time period on the basis of sensor data from at least one specified sensor of the sensor module, provide exercise posture information related to the type of exercise corresponding to the exercise-ready posture, drive a recognition schema for the user's exercise counting corresponding to the exercise-ready posture, perform exercise counting based on the recognition schema, and provide exercise information according to the exercise counting.

According to an embodiment, the processor 120 may operate to recognize exercise counting on the basis of a countable signal based on at least one specified sensor of the sensor module.

According to an embodiment, the processor 120 may operate to recognize an exercise posture corresponding to the recognized exercise-ready posture and, while recognizing the exercise posture, provide exercise posture information related to the recognized exercise-ready posture on the basis of visual images and/or text.

According to an embodiment, the processor 120 may operate to compare the exercise posture with a pre-specified reference exercise posture corresponding to the recognized exercise-ready posture and determine whether to maintain the exercise posture on the basis of a similarity between the exercise posture and the reference exercise posture.

According to an embodiment, the processor 120 may operate to correct the exercise-ready posture when the exercise posture does not substantially match the reference exercise posture, determine a first recognition schema as the recognition schema for counting the user's exercise according to the corrected exercise-ready posture, and continuously accumulate and provide exercise information on the basis of exercise counting based on the determined first recognition schema.

According to an embodiment, the processor 120 may operate to determine a second recognition schema as the recognition schema for counting the user's exercise according to the exercise-ready posture when the exercise posture substantially matches the reference exercise posture, and continuously accumulate and provide exercise information on the basis of exercise counting based on the determined second recognition schema.

According to an embodiment, the processor 120 may operate to determine whether the exercise-ready posture is maintained for a determined time period on the basis of the result of recognizing the exercise-ready posture during the specified time period, and, when the exercise-ready posture is maintained for the determined time period, drive the recognition schema corresponding to the exercise-ready posture.

According to an embodiment, the processor 120 may operate to detect an exercise counting candidate section on the basis of at least one sensor data from at least one specified sensor, filter exercise motion and non-exercise motion for the exercise counting candidate section, calculate the exercise posture at the start and end points of the exercise counting candidate section, and calculate a similarity between the exercise-ready posture and the exercise posture of the exercise counting candidate section.

According to an embodiment, the processor 120 may operate to extract a countable signal through signal processing of at least one specified sensor data and detect an exercise counting candidate section using the extracted countable signal.

According to an embodiment, the processor 120 may operate to extract a countable signal on the basis of sensor data from a specified sensor, detect zero crossing points for the extracted countable signal, detect peaks and valleys within the zero crossing section, determine whether the peaks and valleys satisfy a specified condition on the basis of whether the peaks and valleys pass through a predefined upper boundary and lower boundary, check the sequence in which the countable signal passes through the upper boundary and lower boundary within the zero crossing section when the specified condition is satisfied, and determine the zero crossing section of the countable signal that satisfy the specified condition as the exercise counting candidate section.

According to an embodiment, the processor 120 may operate to perform filtering of exercise counting false recognition based on characteristic parameters for distinguishing between exercise motion and non-exercise motion. According to an embodiment, the characteristic parameters may include the amount of change in acceleration, acceleration peak-valley interval, amount of change in angular velocity, and/or amount of change in atmospheric pressure.

According to an embodiment, the processor 120 may operate to determine that the user is maintaining the exercise posture when the similarity exceeds a specified threshold and update the exercise information accordingly.

According to an embodiment, the processor 120 may operate to determine that the exercise posture is not being maintained when the similarity is equal to or less than the specified threshold and correct the exercise-ready posture.

According to an embodiment, the processor 120 may operate to accumulate the exercise counting candidate section when the similarity is equal to or less than a specified first threshold, determine whether there is a correction history for the exercise-ready posture when the number of candidate sections exceeds a predetermined count, correct the exercise-ready posture when there is no correction history for the exercise-ready posture, and provide guidance on the exercise posture when there is a correction history for the exercise-ready posture.

According to an embodiment, the processor 120 may operate to control the communication circuit to connect with a specified external device via wireless communication upon detecting the exercise start trigger, be configured to transmit visual information related to exercise coaching to the external device, and enable the external device to display the visual information related to exercise coaching.

According to an embodiment, the visual information related to exercise coaching may be configured to be displayed through the display of the electronic device and/or the display of the external device.

Hereinafter, a method of operating the electronic device 101 according to various embodiments will be described in detail. The operations performed by the electronic device 101, according to various embodiments, may be executed by the processor 120, which includes various processing circuitry and/or executable program elements of the electronic device 101. According to an embodiment, the operations performed by the electronic device 101 may be executed by instructions that are stored in the memory 130 and allow the processor 120 to be operated when executed.

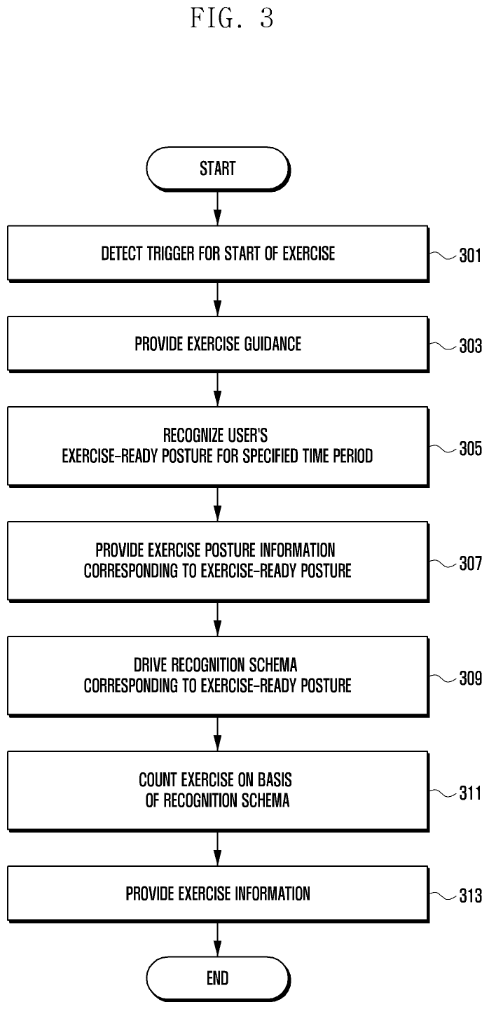

FIG. 3 is a flowchart illustrating a method of operating an electronic device according to an embodiment of the present disclosure.

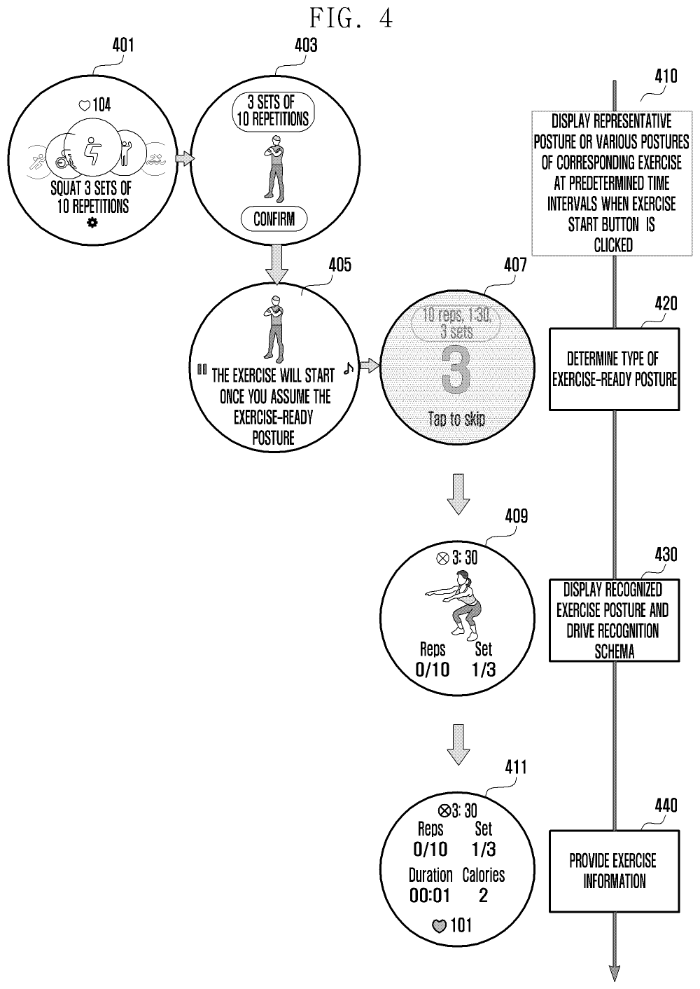

FIG. 4 is a view illustrating an example of a user interface supporting exercise coaching in an electronic device according to an embodiment of the present disclosure.

According to an embodiment, FIG. 3 and FIG. 4 may illustrate an example of supporting exercise coaching for a specified exercise (e.g., squat exercise) by automatically determining the type of exercise-ready posture when the user begins the specified exercise.

In the electronic device (e.g., electronic device 101 in FIG. 1 and/or FIG. 2) according to an embodiment of the present disclosure, a method of supporting exercise coaching may be performed, for example, according to the flowchart illustrated in FIG. 3. The flowchart illustrated in FIG. 3 is merely a flowchart according to an embodiment of an exercise coaching method for the electronic device 101, and the sequence of at least some operations may be modified, performed in parallel, performed as independent operations, or at least some additional operations may be performed complementarily to at least some of the existing operations. According to an embodiment of the present disclosure, operations 301 to 313 may be performed by at least one processor of the electronic device 101 (e.g., processor 120 in FIG. 1 and/or FIG. 2).

As illustrated in FIG. 3, a method performed by the electronic device 101 according to an embodiment (e.g., exercise counting and an information provision method based on exercise counting) may include: detecting an exercise start trigger (operation 301), providing exercise guidance (operation 303), recognizing the user's exercise-ready posture for a specified time period (operation 305), providing exercise posture information corresponding to the exercise-ready posture (operation 307), driving a recognition schema for the user's exercise counting corresponding to the exercise-ready posture (operation 309), performing exercise counting based on the recognition schema (operation 311), and providing exercise information (operation 313).

With reference to FIG. 3, at operation 301, the processor 120 of the electronic device 101 may detect an exercise start trigger. According to an embodiment, the processor 120 may detect a specified user input to trigger the start of exercise. For example, the specified user input may include a voice command (e.g., user utterance of “start XXX exercise”) input and/or a touch input to run a related exercise application based on the operation of the electronic device 101 (e.g., selecting the “start XXX exercise” button). An example of this is illustrated in example screens 401 to 403 and block 410 of FIG. 4.

As illustrated in FIG. 4, in block 410, the processor 120 may display the representative posture or various postures of the corresponding exercise at predetermined time intervals upon detecting a click on the start exercise button. According to an embodiment, the processor 120 may execute an exercise application (e.g., application 146 in FIG. 1) in response to a user input (e.g., clicking the start exercise button). According to an embodiment, the processor 120 may, in response to executing the exercise application, provide a first user interface (e.g., type of exercise selection screen) in a specified structure through the display module (e.g., display module 160 in FIG. 1 and/or display 210 in FIG. 2). For example, the processor 120 may provide the user interface in a carousel structure (e.g., image slide), as illustrated in example screen 401. In an embodiment, the carousel may be an element of the interface, representing a structure that displays a large image in the center of the screen and automatically or in response to user input (e.g., slide or scroll input) displays the next image in the center of the screen.

According to an embodiment, the processor 120 may, in response to a user input (e.g., touching the squat image) selecting a specific exercise (e.g., squat) from the first user interface, provide a second user interface (e.g., exercise start confirmation screen or guidance screen) related to the exercise (e.g., squat exercise) corresponding the user's selection, as illustrated in example screen 403. For example, the processor 120 may provide visual information on the representative posture or various postures of the exercise selected by the user, as well as visual information related to the performance conditions set for the corresponding exercise (e.g., 3 sets of 10 repetitions). According to an embodiment, the processor 120 may detect an exercise start trigger in response to a user input for starting the exercise (e.g., selecting the “confirm” button to start the selected exercise (e.g., squat exercise)) from the second user interface.

At operation 303, the processor 120 may provide exercise guidance. According to an embodiment, the processor 120 may control related constituent elements (e.g., display module 160 and/or speaker) to output specified visual and/or auditory guidance on the basis of detecting an exercise start trigger. According to an embodiment, the processor 120 may provide voice guidance and/or text guidance to the user upon detecting a user input (e.g., click) based on the exercise start button (e.g., squat exercise image), such as “The exercise will begin if you hold the exercise-ready posture for a predetermined time period (e.g., about 3 seconds, about 5 seconds).” An example of this is illustrated in example screens 405 to 407 and block 420 in FIG. 4.

As illustrated in FIG. 4, in block 420, the processor 120 may determine the type of exercise-ready posture. According to an embodiment, the processor 120 may provide first guidance to the user regarding performing the exercise-ready posture, as illustrated in example screen 405. According to an embodiment, the processor 120 may notify the user of an exercise start occasion and provide second guidance related to canceling the exercise start (e.g., count information and skip information), as illustrated in example screen 407. According to an embodiment, the processor 120 may provide voice guidance and/or text guidance to the user, such as “The exercise will begin if you hold the exercise-ready posture for a predetermined time period (e.g., about 3 seconds, about 5 seconds),” and operate to recognize the user's exercise-ready posture and its type during the predetermined time period.

At operation 305, the processor 120 may recognize the user's exercise-ready posture for a specified time period. According to an embodiment, the processor 120 may recognize the user's exercise-ready posture on the basis of sensor data from at least one specified sensor of the sensor module 230 (e.g., acceleration sensor 240, gyro sensor 250, atmospheric pressure sensor 260 in FIG. 2). According to an embodiment, the processor 120 may determine the type of exercise-ready posture for the initial exercise-ready posture through clustering when the user maintains a specific exercise-ready posture for a predetermined time period or more. According to an embodiment, the operation of recognizing the user's initial exercise-ready posture (e.g., determining the type of exercise-ready posture) will be described in detail with reference to the drawings described below.

At operation 307, the processor 120 may provide exercise posture information corresponding to the exercise-ready posture. According to an embodiment, the processor 120 may control the display module 160 to display exercise posture information related to the type of exercise corresponding to the recognized exercise-ready posture (e.g., a specific squat exercise corresponding to the recognized exercise-ready posture among various types of squat exercises) on the basis of visual images and/or text. An example of this is illustrated in example screen 409 and block 430 in FIG. 4.

According to an embodiment, the processor 120 may provide exercise posture information corresponding to the exercise-ready posture on the basis of a mapping table in which exercise posture information is matched to each exercise-ready posture. According to an embodiment, the mapping table may be pre-set in the memory (e.g., memory 130 in FIG. 1 and/or FIG. 2). According to an embodiment, the mapping table may be updated through learning based on the user's exercise. An example of the mapping table according to an embodiment is illustrated in Table 1 below.

TABLE 1

| Exercise- | | | | | |
|---|---|---|---|---|---|
| ready | | | | Type of | Posture |
| posture | Roll | Pitch | Yaw | exercise | information |
| First | Approx. −10 to | Approx. −10 to | Approx. −10 to | First type | First |
| posture | approx. 10 | approx. 10 | approx. 10 | | posture |
| | | | | | information |
| Second | Approx. −60 to | Approx. 10 to | Approx. −10 to | Second type | Second |
| posture | approx. −40 | approx. 30 | approx. 10 | | posture |
| | | | | | information |
| Third | Approx. −85 to | Approx. 0 to | Approx. 70 to | Third type | Third |
| posture | approx. −65 | approx. 20 | approx. 90 | | posture |
| | | | | | information |
| Fourth | Approx. −10 to | Approx. −10 to | Approx. 70 to | Fourth type | Fourth |
| posture | approx. 10 | approx. 10 | approx. 90 | | posture |
| | | | | | information |
| Fifth | Approx. 60 to | Approx. −120 to | Approx. −45 to | Fifth type | Fifth |
| posture | approx. 90 | approx. −100 | approx. 25 | | posture |
| | | | | | information |
| Sixth | Approx. −10 to | Approx. 90 to | Approx. −10 to | Sixth type | Sixth |
| posture | approx. 10 | approx. 110 | approx. 10 | | posture |
| | | | | | information |
| Seventh | Approx. −70 to | Approx. −40 to | Approx. 110 to | Seventh type | Seventh |
| posture | approx. −50 | approx. 20 | approx. 130 | | posture |
| | | | | | information |
| Eighth | Approx. 30 to | Approx. −60 to | Approx. 10 to | Eighth type | Eighth |
| posture | approx. 50 | approx. −40 | approx. 30 | | posture |
| | | | | | information |
| . . . | . . . | . . . | . . . | . . . | . . . |

In an embodiment, Table 1 may illustrate an example of a mapping table for distinguishing the type of exercise (e.g., type of squat exercise) based on an initial exercise-ready posture, while the user wears the electronic device 101 on the user's body (e.g., wrist). For example, the mapping table may have sensor data (e.g., roll, pitch, yaw) of specified sensors (e.g., gyro sensor or attitude sensor) pre-set for each exercise-ready posture (e.g., first posture to eighth posture) representing various types of exercise (e.g., type of squat exercise). According to an embodiment, the processor 120 may recognize an exercise-ready posture, such as a first posture to an eighth posture, on the basis of sensor data from specified sensors. According to an embodiment, the processor 120 may identify a specified exercise type (e.g., a first type to an eighth type) on the basis of the exercise-ready posture. According to an embodiment, the processor 120 may identify posture information (e.g., first posture information to eighth posture information) corresponding to the identified type of exercise, and may provide exercise posture information visually and/or audibly on the basis of the identified posture information.

At operation 309, the processor 120 may drive a recognition schema (or exercise counting recognition algorithm) corresponding to the exercise-ready posture. According to an embodiment, the processor 120 may determine a recognition schema for the user's exercise counting for each exercise-ready posture. For example, the processor 120 may determine an nth recognition schema (or nth exercise counting recognition algorithm) specified for an nth posture, for exercise counting for the exercise according to the nth posture of exercise-ready posture.

An example corresponding to operation 307 and operation 309 is illustrated in example screen 409 and block 430 of FIG. 4.

As illustrated in FIG. 4, in block 430, the processor 120 may drive the recognized exercise posture display and the recognition schema. According to an embodiment, the processor 120 may display exercise posture information related to the type of exercise corresponding to the recognized exercise-ready posture on the basis of visual images and/or text, while internally driving a recognition schema corresponding to the type of exercise.

At operation 311, the processor 120 may perform exercise counting on the basis of the recognition schema. According to an embodiment, the processor 120 may recognize exercise counting on the basis of a countable signal based on at least one specified sensor of the sensor module 230. According to an embodiment, the operation of performing exercise counting is described in detail with reference to the drawings described below.

At operation 313, the processor 120 may provide exercise information. According to an embodiment, the processor 120 may provide exercise information on the basis of auditory (e.g., sound), visual (e.g., display), and/or tactile (e.g., vibration) feedback, so that the user may recognize each time exercise counting is recognized once. According to an embodiment, the processor 120 may provide various types of exercise information, such as exercise counting information and/or healthcare information (e.g., calorie information, heart rate information), by updating this various types of exercise information in proportion to the user's amount of exercise. An example of this is illustrated in example screen 411 and block 440 in FIG. 4.

As illustrated in FIG. 4, in block 440, the processor 120 may provide at least one specified type of exercise information, such as recognized exercise counting information, calorie information, and/or heart rate information.

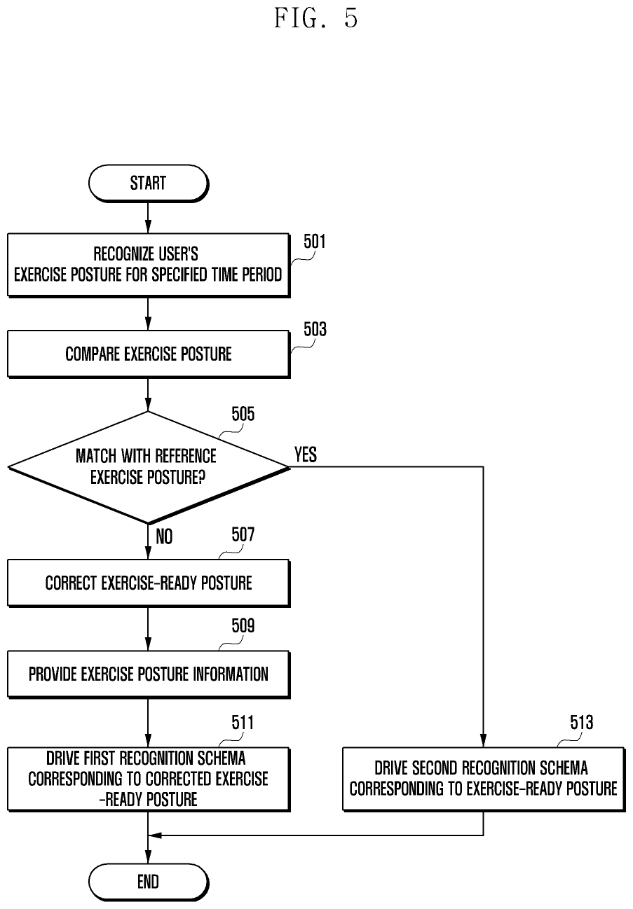

FIG. 5 is a flowchart illustrating a method of operating an electronic device according to an embodiment of the present disclosure.

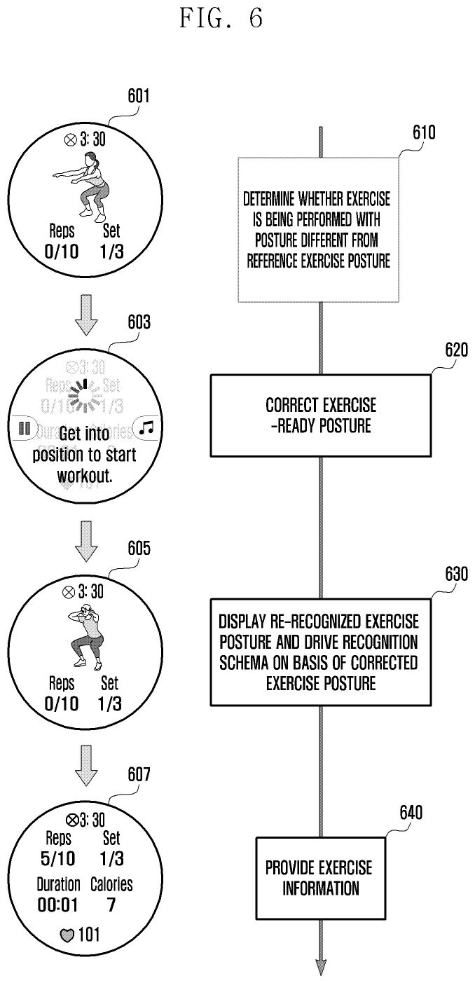

FIG. 6 is a view illustrating an example of a user interface supporting exercise coaching in an electronic device according to an embodiment of the present disclosure.

According to an embodiment, FIG. 5 and FIG. 6 may illustrate an example of identifying a type of exercise-ready posture on the basis of the specified exercise-ready posture of a specified exercise (e.g., squat exercise), and the exercise-ready posture performed by the user, thereby supporting exercise coaching accordingly. For example, FIG. 5 and FIG. 6 may illustrate an example of supporting exercise coaching when the user performs an exercise in a posture different from the specified exercise-ready posture. According to an embodiment, the operations described in FIG. 5 and FIG. 6 may be performed heuristically, for example, in combination with the operations described in FIG. 3 and FIG. 4, or may be heuristically performed as detailed operations of at least a part of the described operations.

In the electronic device 101 according to an embodiment of the present disclosure, a method of supporting exercise coaching may be performed, for example, according to the flowchart illustrated in FIG. 5. The flowchart illustrated in FIG. 5 is merely a flowchart according to an embodiment of an exercise coaching method for the electronic device (e.g., electronic device 101 in FIG. 1 and/or FIG. 2), and the sequence of at least some operations may be modified, performed in parallel, performed as independent operations, or at least some additional operations may be performed complementarily to at least some of the existing operations. According to an embodiment of the present disclosure, operations 501 to 513 may be performed by at least one processor of the electronic device 101 (e.g., processor 120 in FIG. 1 and/or FIG. 2).

As illustrated in FIG. 5, the operation method performed by the electronic device 101 according to an embodiment (e.g., a method of identifying a type of exercise-ready posture) may include: recognizing the user's exercise-ready posture for a specified time period (operation 501), comparing the exercise-ready posture (operation 503), determining whether the recognized exercise-ready posture substantially matches a reference exercise-ready posture (operation 505), correcting the reference exercise-ready posture when the exercise-ready posture is different (operation 507), driving a first recognition schema corresponding to the corrected exercise-ready posture (operation 509), and, when the exercise-ready posture substantially matches, driving a second recognition schema corresponding to the exercise-ready posture (operation 511).

With reference to FIG. 5, at operation 501, the processor 120 of the electronic device 101 may recognize the user's exercise posture for a specified time period. According to an embodiment, the processor 120 may recognize the user's exercise posture on the basis of sensor data from at least one specified sensor (e.g., acceleration sensor 240, gyro sensor 250, atmospheric pressure sensor 260 in FIG. 2) of the sensor module (e.g., sensor module 230 in FIG. 2). According to an embodiment, the processor 120 may monitor the corresponding exercise posture on the basis of the initially recognized exercise-ready posture. According to an embodiment, while recognizing the exercise posture, the processor 120 may provide exercise posture information related to the initially recognized exercise-ready posture on the basis of visual images and/or text. An example of this is illustrated in example screen 601 and block 610 in FIG. 6.

At operation 503, the processor 120 may compare the exercise posture. According to an embodiment, the processor 120 may compare the recognized user's exercise posture with a pre-specified reference exercise posture corresponding to the initially recognized exercise-ready posture. For example, the processor 120 may compare the user's exercise posture with the reference exercise posture according to the initially recognized exercise-ready posture in order to determine whether the user is performing the exercise in the exercise posture corresponding to the initially recognized exercise-ready posture.

At operation 505, the processor 120 may determine whether the exercise posture substantially matches the reference exercise posture. According to an embodiment, the processor 120 may determine whether the exercise posture matches the reference exercise posture on the basis of a similarity between the user's exercise posture and the reference exercise posture.

An example corresponding to operation 501 to operation 505 is illustrated in example screen 601 and block 610 of FIG. 6.

As illustrated in FIG. 6, in block 610, the processor 120 may determine whether the user is performing the exercise in a posture different from the reference exercise posture. According to an embodiment, the processor 120 may recognize the user's exercise posture for a specified time period and determine whether the user is performing the exercise in a posture different from the reference exercise posture. According to an embodiment, while recognizing the exercise posture, the processor 120 may provide exercise posture information related to the initially recognized exercise-ready posture on the basis of visual images and/or text.

At operation 505, when the recognized user's exercise posture does not substantially match the reference exercise posture (e.g., “No” at operation 505), at operation 507, the processor 120 may correct the exercise-ready posture. According to an embodiment, when the exercise posture corresponding to the initially recognized exercise-ready posture differs from the user's exercise posture during the actual exercise, the processor 120 may determine that the user is performing the exercise in a posture different from the initial exercise-ready posture. For example, after the user clicks the start exercise button, there may be instances where the user is simply standing still without assuming the ready posture, or intentionally performing the exercise in a different posture as opposed to the exercise-ready posture. For example, when the user is attempting to start a squat exercise while placing a barbell on the shoulders, the electronic device 101 may calculate the initial exercise-ready posture according to the corresponding exercise-ready posture. However, there may be a case where the user later performs the exercise in a different posture at an occasion when the user performs the actual exercise (e.g., holding a kettle bell between the legs while performing a squat).

In such cases, when the exercise posture based on the initial exercise-ready posture differs from the user's actual exercise posture, it may not be considered an exercise motion, and exercise counting may not be recognized. For example, at the beginning of the exercise, exercise counting may not be recognized normally. Accordingly, according to an embodiment of the present disclosure, when the recognized user's exercise posture does not substantially match the reference exercise posture, the processor 120 may correct the initially recognized exercise-ready posture. According to an embodiment, during correction of the exercise-ready posture, the processor 120 may perform an operation to correct the reference exercise-ready posture to an exercise-ready posture corresponding to the user's exercise posture while displaying a loading animation. An example of this is illustrated in example screen 603 and block 620 in FIG. 6.

As illustrated in FIG. 6, in block 620, the processor 120 may correct the reference exercise-ready posture to an exercise-ready posture corresponding to the user's exercise posture. During the correction of the exercise-ready posture, the processor 120 may display a loading animation and related information (e.g., “Get into position to start workout”).

At operation 509, the processor 120 may provide exercise posture information corresponding to the corrected exercise-ready posture. According to an embodiment, the processor 120 may control the display module 160 to display exercise posture information related to the type of exercise corresponding to the corrected exercise-ready posture)(e.g., a specific squat exercise corresponding to the recognized exercise-ready posture among various types of squat exercises) on the basis of visual images and/or text. An example of this is illustrated in example screen 605 and block 630 in FIG. 6. As illustrated in FIG. 6, in block 630, the processor 120 may drive the recognition schema on the basis of the re-recognized exercise posture display and the corrected exercise-ready posture. First exercise posture information corresponding to the initially recognized exercise-ready posture (e.g., example screen 601) may be changed to second exercise posture information corresponding to the corrected exercise-ready posture (e.g., example screen 605) and provided accordingly. For example, the processor 120 may update and provide a type of user's exercise posture on the screen according to the corrected exercise-ready posture.

At operation 511, the processor 120 may drive the first recognition schema corresponding to the corrected exercise-ready posture. According to an embodiment, the processor 120 may determine the first recognition schema as the recognition schema for counting the user's exercise according to the corrected exercise-ready posture. For example, the processor 120 may determine an mth recognition schema (or mth exercise counting recognition algorithm) specified for an mth posture, for exercise counting for the exercise according to the mth posture of corrected exercise-ready posture.

An example corresponding to operation 509 and operation 511 is illustrated in example screen 605 and block 630 of FIG. 6.

As illustrated in FIG. 6, in block 630, the processor 120 may drive the recognition schema on the basis of the re-recognized exercise posture display and the corrected exercise-ready posture. According to an embodiment, the processor 120 may change and provide the first exercise posture information corresponding to the initially recognized exercise-ready posture (e.g., example screen 601) as the second exercise posture information corresponding to the corrected exercise-ready posture (e.g., example screen 605). For example, the processor 120 may update and provide a type of user's exercise posture on the screen according to the corrected exercise-ready posture. According to an embodiment, the processor 120 may display the corrected type of exercise posture while internally driving a recognition schema (e.g., first recognition schema) corresponding to the type of exercise.

According to an embodiment, the processor 120 may provide exercise information related to exercise counting based on the determined first recognition schema. For example, on the basis of the correction of the exercise-ready posture, when the exercise is counted normally, the processor 120 may provide exercise information on the basis of auditory, visual, and/or tactile information each time exercise counting is recognized once, allowing the user to perceive the exercise information.

According to an embodiment, the processor 120 may accumulate and provide various types of exercise information, such as exercise counting information and/or healthcare information, continuously in sequence with the previous exercise information. An example of this is illustrated in example screen 607 and block 640 in FIG. 6.

As illustrated in FIG. 6, in block 640, the processor 120 may provide at least one specified type of exercise information, such as recognized exercise counting information, calorie information, and/or heart rate information. According to an embodiment, the processor 120 may not start exercise counting from the re-recognized exercise posture, but instead continue counting from the exercise counting of the previous exercise posture, updating the exercise information according to the previous exercise posture (e.g., example screen 411 in FIG. 4) and providing the continuous exercise information (e.g., example screen 607 in FIG. 6).

At operation 505, when the recognized user's exercise posture substantially matches the reference exercise posture (e.g., “Yes” at operation 505), at operation 513, the processor 120 may drive the second recognition schema corresponding to the initially recognized exercise-ready posture. According to an embodiment, the processor 120 may determine the second recognition schema, different from the first recognition schema, for counting the user's exercise based on the initially recognized exercise-ready posture. For example, the processor 120 may determine an nth recognition schema (or nth exercise counting recognition algorithm) specified for an nth posture, for exercise counting for the exercise according to the nth posture of exercise-ready posture.

According to an embodiment, the processor 120 may provide exercise information related to exercise counting based on the determined second recognition schema. For example, when the exercise is counted normally, the processor 120 may provide exercise information on the basis of auditory, visual, and/or tactile information each time exercise counting is recognized once, allowing the user to perceive the exercise information,

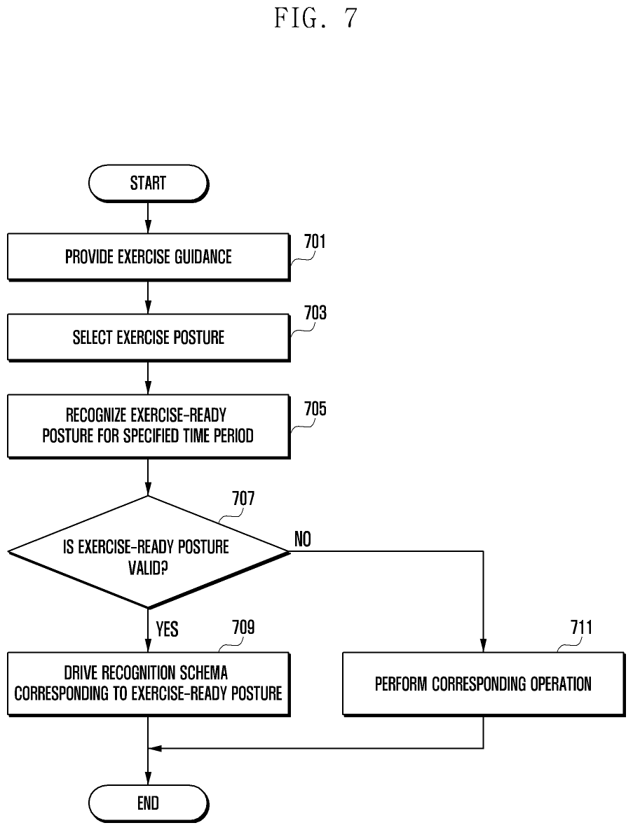

FIG. 7 is a flowchart illustrating a method of operating an electronic device according to an embodiment of the present disclosure.

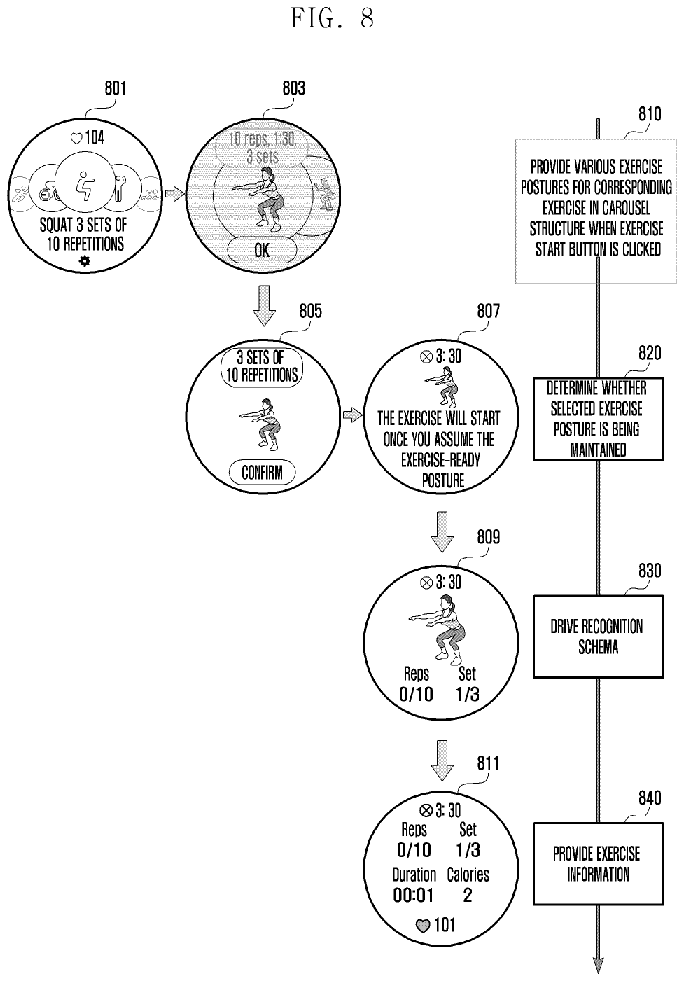

FIG. 8 is a view illustrating an example of a user interface supporting exercise coaching in an electronic device according to an embodiment of the present disclosure.

According to an embodiment, FIG. 7 and FIG. 8 may illustrate an example of identifying a type of exercise-ready posture on the basis of the exercise-ready posture specified by the user, thereby supporting exercise coaching accordingly. For example, FIG. 7 and FIG. 8 may illustrate an example of providing information on various postures supportable for a specified exercise (e.g., exercise guidance) when the user starts a specified exercise and supporting exercise coaching based on the type of exercise-ready posture corresponding to the information selected by the user. According to an embodiment, the operations described in FIG. 7 and FIG. 8 may be performed heuristically, for example, in combination with the operations described in FIG. 3 to FIG. 6, or may be heuristically performed as detailed operations of at least a part of the described operations.

In the electronic device (e.g., electronic device 101 in FIG. 1 and/or FIG. 2) according to an embodiment of the present disclosure, a method of supporting exercise coaching may be performed, for example, according to the flowchart illustrated in FIG. 7. The flowchart illustrated in FIG. 7 is merely a flowchart according to an embodiment of an exercise coaching method for the electronic device 101, and the sequence of at least some operations may be modified, performed in parallel, performed as independent operations, or at least some additional operations may be performed complementarily to at least some of the existing operations. According to an embodiment of the present disclosure, operations 701 to 711 may be performed by at least one processor of the electronic device 101 (e.g., processor 120 in FIG. 1 and/or FIG. 2).

As illustrated in FIG. 7, the operation method performed by the electronic device 101 according to an embodiment (e.g., a method of identifying a type of exercise-ready posture) may include: providing exercise guidance (operation 701), selecting an exercise posture (operation 703), recognizing the exercise-ready posture for a specified time period (operation 705), determining whether the exercise-ready posture is valid (operation 707), driving a recognition schema corresponding to the exercise-ready posture when the exercise-ready posture is valid (operation 709), and, when the exercise-ready posture is not valid, performing the corresponding operation (e.g., performing exercise-ready posture correction and/or providing correction guidance) (operation 711).

With reference to FIG. 7, at operation 701, the processor 120 of the electronic device 101 may provide exercise guidance. According to an embodiment, the processor 120 may provide specified exercise guidance (e.g., exercise type selection screen) on the basis of detecting an exercise start trigger. According to an embodiment, when detecting a user input (e.g., click) based on an exercise start button, the processor 120 may provide exercise guidance (e.g., exercise posture type selection screen) related to various exercise postures for a specified exercise (e.g., squat exercise) to the user. An example of this is illustrated in example screens 801 to 803 and block 810 in FIG. 8.

As illustrated in FIG. 8, in block 810, when detecting a click on the start exercise button, the processor 120 may provide various exercise postures for the corresponding exercise in a carousel structure. According to an embodiment, the processor 120 may execute an exercise application (e.g., application 146 in FIG. 1) in response to a user input (e.g., clicking the start exercise button). According to an embodiment, in response to executing an exercise application, the processor 120 may provide a first user interface (e.g., exercise type selection screen) in a carousel structure through a display module (e.g., display module 160 in FIG. 1 and/or display 210 in FIG. 2), as illustrated in example screen 801.

According to an embodiment, the processor 120 may provide guidance on various exercise postures to the user, as illustrated in example screen 803, in response to a user input selecting a specific exercise (e.g., squat) on the first user interface (e.g., touching a squat image). According to an embodiment, the exercise guidance may include information on various exercise postures supportable for a specified exercise (e.g., squat exercise). According to an embodiment, the processor 120 may provide exercise guidance (e.g., exercise posture type selection screen) in a specified structure through the display module 160. For example, as illustrated in example screen 803, the processor 120 may provide exercise guidance in a carousel structure (e.g., image slide). In an embodiment, the carousel may be an element of the interface, representing a structure that displays a large image in the center of the screen and automatically or in response to user input (e.g., slide or scroll input) displays the next image in the center of the screen.

At operation 703, the processor 120 may select an exercise posture. According to an embodiment, the processor 120 may select an exercise posture corresponding to a user input (e.g., click) based on the provided exercise guidance. According to an embodiment, the user may start the exercise by selecting the desired exercise posture from the exercise posture type selection screen.

At operation 705, the processor 120 may recognize the exercise-ready posture for a specified time period. The processor 120 may recognize the user's exercise-ready posture on the basis of sensor data from at least one specified sensor of the sensor module 230 (e.g., acceleration sensor 240, gyro sensor 250, atmospheric pressure sensor 260 in FIG. 2). According to an embodiment, when a specific exercise posture (e.g., squat posture) for a specified exercise is selected by the user, the processor 120 may recognize the user's exercise-ready posture for a specified time period.

At operation 707, the processor 120 may determine whether the exercise-ready posture is valid. According to an embodiment, the processor 120 may determine whether the user maintains the specified exercise-ready posture for a predetermined time period or longer, on the basis of the result of recognizing the exercise-ready posture for a specified time period.

An example corresponding to operation 703 to operation 707 is illustrated in example screens 805 to 807 and block 820 in FIG. 8.

As illustrated in FIG. 8, in block 820, the processor 120 may determine whether the selected exercise posture is being maintained. According to an embodiment, the processor 120 may provide first guidance to the user regarding performing the exercise-ready posture, as illustrated in example screen 805. According to an embodiment, in response to a user input selecting an exercise posture, the processor 120 may provide first guidance related to the selected exercise (e.g., exercise start confirmation screen or guidance screen), as illustrated example screen 805. For example, the processor 120 may provide visual information related to the confirmation of the exercise selected by the user and the performance conditions set for the corresponding exercise (e.g., 3 sets of 10 repetitions). According to an embodiment, in response to a user input for exercise start from the first guidance (e.g., selecting the “Confirm” button to start the selected exercise (e.g., squat exercise)), the processor 120 may provide second guidance, as illustrated in example screen 807. According to an embodiment, the processor 120 may provide voice guidance and/or text guidance to the user, such as “The exercise will start once you assume the exercise-ready posture.” According to an embodiment, the processor 120 may provide voice guidance and/or text guidance to the user and determine whether the user's exercise posture is maintained for a predetermined time period.

At operation 707, when the exercise-ready posture is valid (e.g., “Yes” at operation 707), at operation 709, the processor 120 may drive a recognition schema (or exercise counting recognition algorithm) corresponding to the exercise-ready posture. An example of this is illustrated in example screen 809 and block 830 in FIG. 8.

As illustrated in FIG. 8, in block 830, the processor 120 may provide exercise posture information related to the type of exercise corresponding to the recognized exercise-ready posture on the basis of visual images and/or text. According to an embodiment, the processor 120 may identify posture information (e.g., first posture information to eighth posture information in Table 1) corresponding to the type of exercise, and may provide exercise posture information visually and/or audibly on the basis of the identified posture information.

According to an embodiment, the processor 120 may determine a recognition schema for exercise counting for each exercise-ready posture. For example, the processor 120 may display exercise posture information related to the type of exercise on the basis of visual images and/or text, while internally driving a recognition schema corresponding to the type of exercise. For example, the processor 120 may determine an nth recognition schema (or nth exercise counting recognition algorithm) specified for an nth posture, for exercise counting for the exercise according to the nth posture of exercise-ready posture.

According to an embodiment, the processor 120 may perform exercise counting on the basis of the determined recognition schema. According to an embodiment, the processor 120 may provide exercise information on the basis of auditory (e.g., sound), visual (e.g., display), and/or tactile (e.g., vibration) feedback, so that the user may recognize each time exercise counting is recognized once. According to an embodiment, the processor 120 may provide various types of exercise information, such as exercise counting information and/or healthcare information (e.g., calorie information, heart rate information), by updating this various types of exercise information in proportion to the user's amount of exercise. An example of this is illustrated in example screen 811 and block 840 in FIG. 8.

As illustrated in FIG. 8, in block 840, the processor 120 may provide at least one specified type of exercise information, such as recognized exercise counting information, calorie information, and/or heart rate information.

At operation 707, when the exercise-ready posture is not valid (e.g., “No” at operation 707), at operation 711, the processor 120 may perform the corresponding operation. According to an embodiment, the processor 120 may perform various specified operations, such as correcting the exercise-ready posture and/or providing guidance indicating that the exercise posture is incorrect.

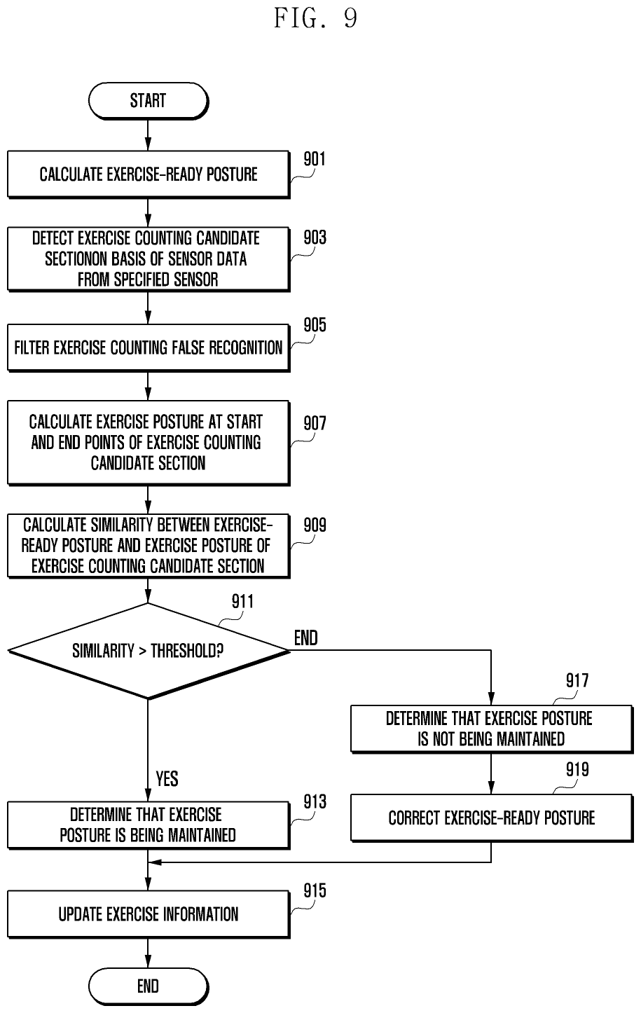

FIG. 9 is a flowchart illustrating a method of operating an electronic device according to an embodiment of the present disclosure.

According to an embodiment, FIG. 9 may illustrate an example of a method of improving the performance of exercise counting recognition for a specified exercise. According to an embodiment, the operations described in FIG. 9 may be performed heuristically, for example, in combination with the operations described in FIG. 3 to FIG. 8, or may be heuristically performed as detailed operations of at least a part of the described operations.

In the electronic device (e.g., electronic device 101 in FIG. 1 and/or FIG. 2) according to an embodiment of the present disclosure, the method of improving the performance of exercise counting recognition may be performed, for example, according to the flowchart illustrated in FIG. 9. The flowchart illustrated in FIG. 9 is merely a flowchart according to an embodiment of improving the performance of exercise counting recognition for the electronic device 101, and the sequence of at least some operations may be modified, performed in parallel, performed as independent operations, or at least some additional operations may be performed complementarily to at least some of the existing operations. According to an embodiment of the present disclosure, operations 901 to 919 may be performed by at least one processor of the electronic device 101 (e.g., processor 120 in FIG. 1 and/or FIG. 2).

As illustrated in FIG. 9, the operation method performed by the electronic device 101 according to an embodiment (e.g., a method of improving the recognition performance of exercise counting) may include: calculating the exercise-ready posture (operation 901), detecting an exercise counting candidate section on the basis of sensor data from specified sensors (operation 903), performing exercise counting false recognition filtering (operation 905), calculating the exercise posture at a start point and end point of the exercise counting candidate section (operation 907), calculating a similarity between the exercise-ready posture and the exercise posture of the exercise counting candidate section (operation 909), determining whether the similarity exceeds a specified threshold (operation 911), when the similarity exceeds the specified threshold, determining that the exercise posture is being maintained (operation 913), updating exercise information (operation 915), when the similarity does not exceed the specified threshold (e.g., equal to or less than the specified threshold), determining that the exercise posture is not being maintained (operation 917), correcting the exercise-ready posture (operation 919), and updating the exercise information on the basis of the exercise counting according to the corrected exercise-ready posture (operation 915).

With reference to FIG. 9, at operation 901, the processor 120 of the electronic device 101 may calculate the exercise-ready posture. According to an embodiment, when no acceleration movement occurs and no other input, such as touch, is detected during a predetermined time period, the processor 120 may determine that the user is in a ready posture and calculate the initial exercise-ready posture.

At operation 903, the processor 120 may detect an exercise counting candidate section on the basis of at least one sensor data from at least one specified sensor. According to an embodiment, the processor 120 may extract a countable signal through signal processing of at least one specified sensor data. According to an embodiment, the processor 120 may detect an exercise counting candidate section using the extracted countable signal. According to an embodiment, the operation of detecting an exercise counting candidate section is described in detail with reference to the drawings provided below.

At operation 905, the processor 120 may perform exercise counting false recognition filtering. According to an embodiment, the processor 120 may perform filtering to distinguish between exercise motions and non-exercise motions for the exercise counting candidate section. According to an embodiment, the processor 120 may evaluate whether the exercise counting candidate section corresponds to an actual exercise motion or a non-exercise motion similar to an exercise motion, and perform filtering of exercise counting false recognition. According to an embodiment, the characteristic parameters for distinguishing (or evaluating) between actual exercise motion and non-exercise motion may include, for example, the amount of change in acceleration, the acceleration peak-valley interval, the amount of change in angular velocity, and/or the amount of change in atmospheric pressure. According to an embodiment, the amount of change in each parameter may be calculated as a peak-to-peak (p2p) within the exercise counting candidate section. According to an embodiment, the operation of performing exercise counting false recognition filtering is described in detail with reference to the drawings provided blew.

At operation 907, the processor 120 may calculate the exercise posture at the start point and end point of the exercise counting candidate section. According to an embodiment, the processor 120 may calculate the exercise posture at the start point and end point of the exercise counting candidate section. For example, the user's posture may slightly be disrupted during exercise, but conventional systems do not consider the case that the posture is disrupted during exercise as an exercise motion, resulting in the disrupted motion not being counted. According to an embodiment of the present disclosure, by excluding any posture disruption occurring during the user's exercise process and checking the exercise posture at the start and end points of a single exercise repetition motion, it is possible to ensure that the exercise is counted normally, even if the user's posture is disrupted during the exercise. For example, in conventional systems, since the exercise posture is continuously monitored from the start occasion to the end occasion of the exercise, even if slight disruption occurs in posture during the exercise, not allowing the disrupted exercise to be counted. However, since the user's exercise posture may be partially disrupted during an exercise motion depending on the circumstances, in an embodiment of the present disclosure, it is possible to check the exercise posture at the start and end points of a single exercise repetition motion, regardless of whether the user's posture is correct or disrupted during the exercise. In an embodiment, a single exercise repetition motion may represent, for example, one repetition of the user performing the motion of squatting down and standing up in a squat exercise.

At operation 909, the processor 120 may calculate the similarity between the exercise-ready posture and the exercise posture within the exercise counting candidate section. According to an embodiment, the processor 120 may calculate the similarity between the initial exercise-ready posture and the posture within the exercise counting candidate section to determine whether the exercise posture is being maintained. According to an embodiment, the operation of determining whether the exercise posture is being maintained is described in detail with reference to the drawings provided below.

At operation 911, the processor 120 may determine whether the similarity exceeds the threshold.

At operation 911, when the similarity exceeds the threshold (e.g., “Yes” at operation 911), at operation 913, the processor 120 may determine that the exercise posture is being maintained. According to an embodiment, when the similarity of the exercise posture exceeds a predetermined threshold, the processor 120 may determine that the user is maintaining the exercise posture properly while exercising. According to an embodiment, the similarity may be calculated using various similarity coefficients, such as cosine similarity and/or Pearson correlation coefficient. According to an embodiment, the operation of determining whether the exercise posture is being maintained is described in detail with reference to the drawings provided below.

At operation 915, the processor 120 may update exercise information. According to an embodiment, the processor 120 may update exercise information (e.g., exercise repetitions, calories, and/or heart rate information). According to an embodiment, when the similarity of the exercise posture is equal to or greater than a predetermined threshold, the processor 120 may determine that the user is maintaining the exercise posture properly while performing the exercise, and may update exercise information on the corresponding exercise, such as exercise count, calories, and/or heart rate.

At operation 911, when the similarity is equal to or less than the threshold (e.g., “No” at operation 911), at operation 917, the processor 120 may determine that the exercise posture is not being maintained. According to an embodiment, when the similarity of the exercise posture is equal to or less than a predetermined threshold, for example, cases where the user is performing the exercise in a posture different from the initial exercise-ready posture, may be included. For example, the cases where the user performs the exercise in a posture different from the initial exercise-ready posture may include cases where the exercise-ready posture was previously calculated by the processor 120 before the user assumed the exercise-ready posture, or where the user intentionally performs the exercise in a different posture from the exercise-ready posture initially taken.

According to an embodiment, a case where the exercise-ready posture is calculated before the user assumes the exercise-ready posture, for example, may be a case where the user remains in a same posture with no motion, such as after clicking the exercise start button and standing still. This case may be calculated as the exercise-ready posture. In this case, when the user actually performs the exercise, the initial exercise-ready posture may differ from the actual exercise posture, resulting in a low similarity being calculated for the exercise posture.

According to an embodiment, when the user intentionally performs the exercise in a posture different from the initial exercise-ready posture, for example, when the user intends to perform a squat exercise with a barbell on the shoulders, the initial exercise-ready posture may be calculated based on the corresponding exercise posture. However, when the user actually performs the squat exercise while holding a kettle bell between the legs during the exercise occasion, the initial exercise-ready posture may differ from the actual exercise posture, leading to a low similarity being calculated for the exercise posture.

At operation 919, the processor 120 may correct the exercise-ready posture. According to an embodiment, when the similarity of the exercise posture is equal to or less than a predetermined threshold, the operation of correcting the exercise-ready posture is described in detail with reference to the drawings provided below.

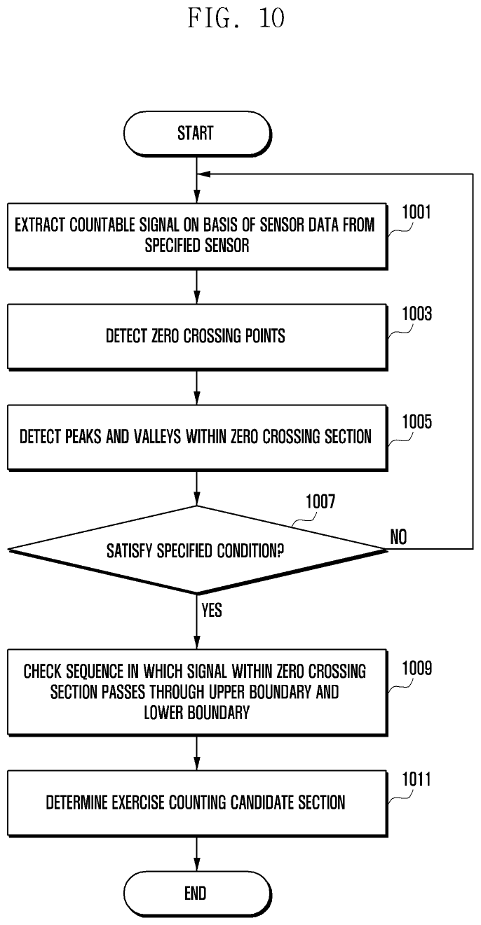

FIG. 10 is a flowchart illustrating a method of operating an electronic device according to an embodiment of the present disclosure.

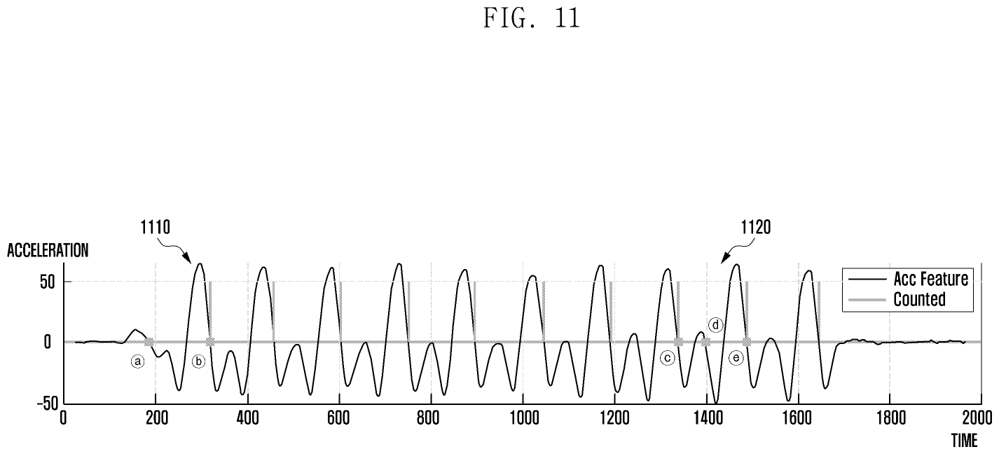

FIG. 11 is a reference view for describing exercise counting candidate section detection according to an embodiment of the present disclosure.

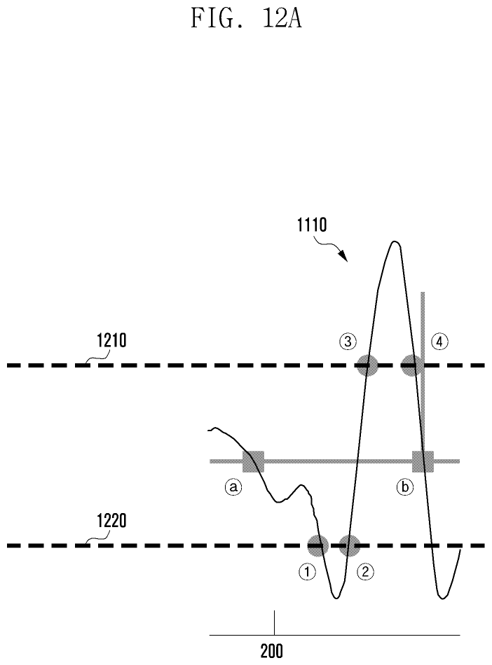

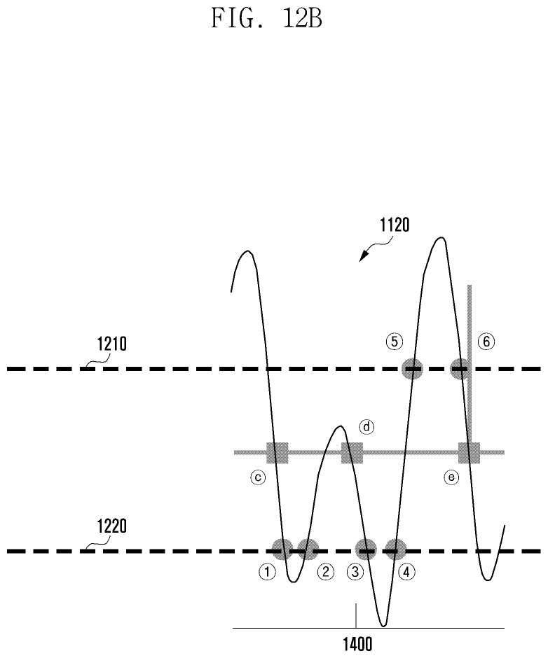

FIG. 12A and FIG. 12B are reference views for describing exercise counting candidate section detection according to an embodiment of the present disclosure.

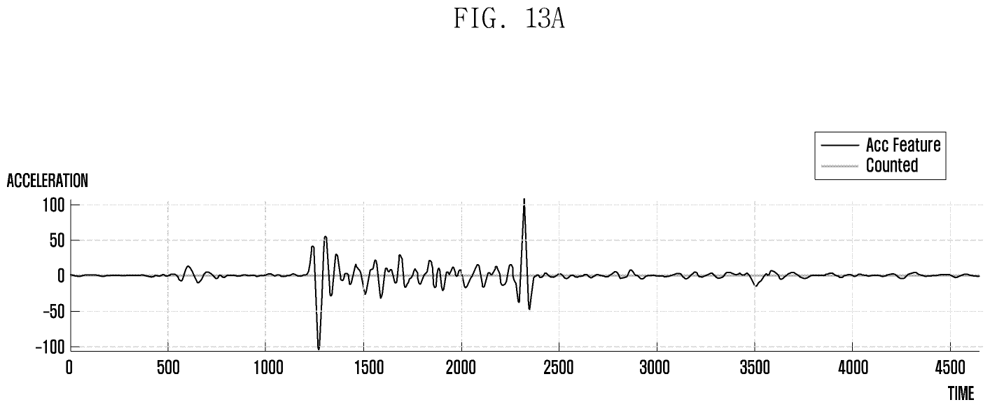

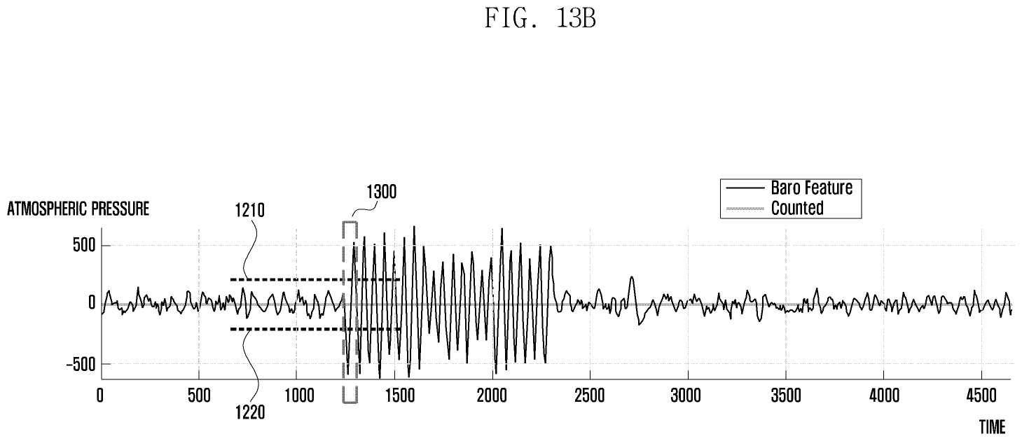

FIG. 13A and FIG. 13B are reference views for describing exercise counting candidate section detection according to an embodiment of the present disclosure.

According to an embodiment, FIG. 10 may illustrate an example of a method of detecting an exercise counting candidate section to improve exercise recognition performance for a specified exercise. According to an embodiment, the operations described in FIG. 10 may be performed heuristically, for example, in combination with the operations described in FIG. 3 to FIG. 9, or may be heuristically performed as detailed operations of at least a part of the described operations.

In the electronic device (e.g., electronic device 101 in FIG. 1 and/or FIG. 2) according to an embodiment of the present disclosure, the method of detecting an exercise counting candidate section to improve the recognition performance of exercise counting may be performed, for example, according to the flowchart illustrated in FIG. 10. The flowchart illustrated in FIG. 10 is merely a flowchart according to an embodiment of improving the performance of exercise counting recognition for the electronic device 101, and the sequence of at least some operations may be modified, performed in parallel, performed as independent operations, or at least some additional operations may be performed complementarily to at least some of the existing operations. According to an embodiment of the present disclosure, operations 1001 to 1011 may be performed by at least one processor of the electronic device 101 (e.g., processor 120 in FIG. 1 and/or FIG. 2).

As illustrated in FIG. 10, the operation method performed by the electronic device 101 according to an embodiment (e.g., a method of detecting an exercise counting candidate section) may include: extracting a countable signal on the basis of sensor data from a specified sensor (operation 1001), detecting a zero crossing point (ZC point) (operation 1003), detecting peaks and valleys within a zero crossing section (operation 1005), determining whether the peaks and valleys satisfy specified conditions (operation 1007), when the peaks and valleys satisfy the specified conditions, checking the sequence of the countable signal within the zero crossing section passing through an upper boundary and a lower boundary (operation 1009), and determining an exercise counting candidate section (operation 1011).

With reference to FIG. 10, at operation 1001, the processor 120 of the electronic device 101 may extract a countable signal on the basis of sensor data from a specified sensor. According to an embodiment, the processor 120 may extract a countable signal through signal processing of an acceleration signal from an acceleration sensor (e.g., acceleration sensor 240 in FIG. 2). According to an embodiment, the processor 120 may apply a low pass filter (LPF) to the acceleration signal, then extract a sliding window summing (SWS) differential signal for a single vector magnitude (SVM) signal, and use this as the countable signal. According to an embodiment, the processor 120 may extract a countable signal through signal processing of an atmospheric pressure signal from an atmospheric pressure sensor (e.g., atmospheric pressure sensor 260 in FIG. 2). According to an embodiment, the processor 120 may apply a low pass filter (LPF) to the atmospheric pressure signal, then extract a sliding window summing (SWS) differential signal, and use this as the countable signal.

At operation 1003, the processor 120 may detect a zero crossing point. According to an embodiment, the processor 120 may detect zero crossing points in the extracted countable signal. According to an embodiment, the processor 120 may detect zero crossing points in the countable signal based on the acceleration signal and/or the atmospheric pressure signal.

At operation 1005, the processor 120 may detect the peaks and valleys within the zero crossing section. According to an embodiment, the processor 120 may extract peak and valley values to check whether the countable signal within the zero crossing section passes through the upper and lower boundaries and to check the sequence of passing through each of these boundaries.

At operation 1007, the processor 120 may determine whether the peaks and valleys satisfy the specified conditions. According to an embodiment, the processor 120 may check whether the peak and valley values pass through the predefined upper boundary and lower boundary.

At operation 1007, when the specified conditions are not satisfied (e.g., “No” at operation 1007), the processor 120 may proceed to operation 1001 and perform the subsequent operations starting from operation 1001.

At operation 1007, when the specified conditions are satisfied (e.g., “Yes” at operation 1007), at operation 1009, the processor 120 may check the sequence in which the countable signal within the zero crossing section passes through the upper boundary and lower boundary.

At operation 1011, the processor 120 may determine the exercise counting candidate section. According to an embodiment, when the specified conditions are satisfied in the countable signal based on acceleration or when the specified conditions are satisfied in the countable signal based on atmospheric pressure, the processor 120 may operate to determine the corresponding zero crossing section as an exercise counting candidate section.

The example operations according to FIG. 10 are described with reference to FIG. 11 to FIG. 13B.

According to an embodiment, the graph illustrated in FIG. 11 represents an example of an acceleration-based countable signal when the user performs a specified exercise (e.g., squat exercise) ten repetitions in an exercise posture with hands clasped in front of the chest. According to an embodiment, in FIG. 11, the x-axis may represent time (seconds), while the y-axis may represent acceleration (m/s²).

With reference to FIG. 11, the processor 120 may detect a point where the countable signal changes from positive (+) to negative (−) as a zero crossing point. For example, in the graph of FIG. 11, points \textcircled{a} to \textcircled{e} may be detected as zero crossing points. According to an embodiment, detecting an exercise counting candidate section may involve detecting a zero crossing section as a single candidate section. For example, sections \textcircled{a} to \textcircled{b}, \textcircled{c} to \textcircled{d}, and \textcircled{d} to \textcircled{e} may each be detected as exercise counting candidate sections. However, when the exercise counting candidate section is detected in this manner, for the section \textcircled{c} to \textcircled{e}, the exercise counting candidate section may be falsely detected as if the user performed the exercise twice, even though the user only performed the exercise once.

Accordingly, according to an embodiment of the present disclosure, to detect the exercise counting candidate section, as illustrated in FIG. 12A and FIG. 12B, whether the signal within the exercise counting candidate section passes through a predetermined upper boundary 1210 and a predetermined lower boundary 1220, as well as the sequence of passing through each boundary 1210 and 1220, may be checked, thereby enabling more accurate detection of the exercise counting candidate section.

With reference to FIG. 12A, FIG. 12A is an enlarged reference view of a portion 1110 that includes the section \textcircled{a} to \textcircled{b} illustrated in FIG. 11, representing an example of a general case. For example, as illustrated in FIG. 12A, the signal may pass through points \textcircled{1} and \textcircled{2} in sequence relative to the lower boundary 1220, and subsequently pass through points \textcircled{3} and \textcircled{4} in sequence relative to the upper boundary 1210. In this manner, when the signal within the section \textcircled{a} to \textcircled{b} passes through the boundaries 1210 and 1220 in sequence, specifically, passes through points \textcircled{1} and \textcircled{2} of the lower boundary 1220, and points \textcircled{3} and \textcircled{4} of the upper boundary 1210 each once, then the section \textcircled{a} to \textcircled{b} may be detected as a single exercise counting candidate section.

With reference to FIG. 12B, FIG. 12B is an enlarged reference view of a portion 1120 that includes the section \textcircled{c} to \textcircled{e} illustrated in FIG. 11, representing an example of a non-general case. For example, FIG. 12B may illustrate a case where the signal passes through at least one of the upper boundary 1210 or the lower boundary 1220 twice. For example, as illustrated in FIG. 12B, the signal may pass through points \textcircled{1} and \textcircled{2}, as well as points \textcircled{3} and \textcircled{4}, in sequence relative to the lower boundary 1220, and subsequently pass through points \textcircled{5} and \textcircled{6} in sequence relative to the upper boundary 1210. In this manner, when the signal within the section \textcircled{c} to \textcircled{e} passes twice through points \textcircled{1} and \textcircled{2}, as well as points \textcircled{3} and \textcircled{4}, of the lower boundary 1220, and then passes through points \textcircled{5} and \textcircled{6} of the upper boundary 1210, the section \textcircled{c} to \textcircled{e}, which sequentially passes through all boundaries 1210 and 1220 from \textcircled{1} to \textcircled{6}, may be detected as a single exercise counting candidate section.

According to an embodiment, the graphs illustrated in FIG. 13A and FIG. 13B represent examples of acceleration-based and atmospheric pressure-based countable signals, when the user performs a squat exercise slowly. According to an embodiment, in FIG. 13A, the x-axis represents time (seconds), while the y-axis represents acceleration (m/s²). According to an embodiment, in FIG. 13B, the x-axis represents time (seconds), while the y-axis represents atmospheric pressure (hPa).

With reference to FIG. 13A and FIG. 13B, generally, due to the characteristics of the acceleration sensor 240, when the user performs a squat exercise slowly, there may be cases where the changes in the sensor data (e.g., acceleration signal) from the acceleration sensor 240 are not clearly noticeable. Accordingly, according to an embodiment of the present disclosure, when detecting an exercise counting candidate section, not only the sensor data from the acceleration sensor 240 but also the sensor data from the atmospheric pressure sensor 260 (e.g., atmospheric pressure signal) may be checked together.

According to an embodiment, the method of detecting an exercise counting candidate section from the atmospheric pressure-based countable signal may be substantially identical to the acceleration-based method described above with reference to FIG. 11 to FIG. 12B. According to an embodiment, in FIG. 13A and FIG. 13B, a portion 1300 marked with a dashed line in the atmospheric pressure-based countable signal passes through the predetermined upper boundary 1210 and predetermined lower boundary 1220, and the corresponding section may be detected as an exercise counting candidate section.

According to an embodiment of the present disclosure, the exercise counting candidate section may be detected when either the method using acceleration or the method using atmospheric pressure satisfies the specified conditions. According to an embodiment of the present disclosure, the exercise counting candidate section may also be detected when both the method using acceleration and the method using atmospheric pressure satisfy the specified conditions.

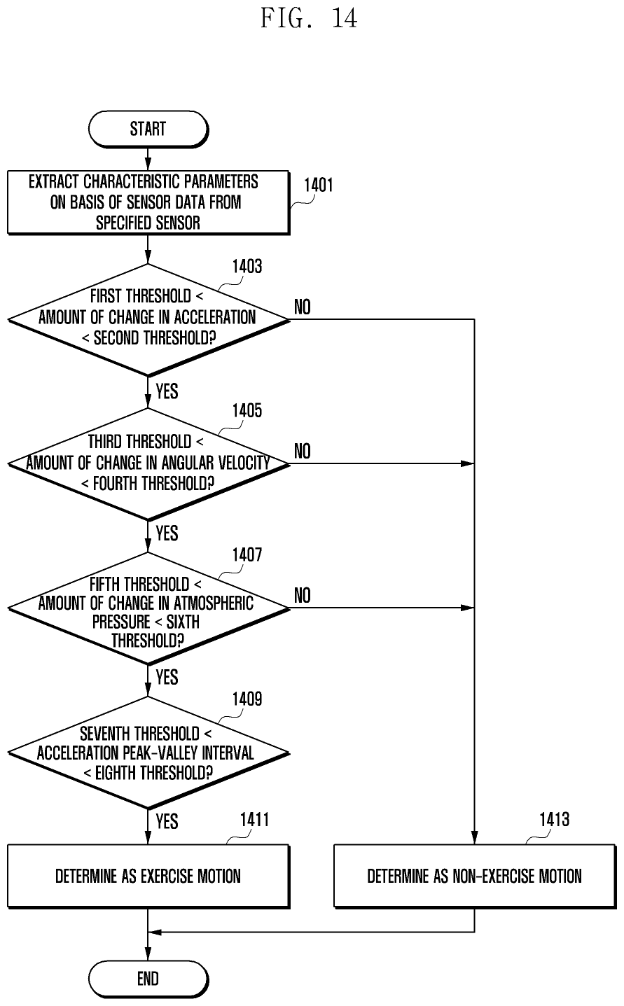

FIG. 14 is a flowchart illustrating a method of operating an electronic device according to an embodiment of the present disclosure.

According to an embodiment, FIG. 14 may illustrate an example of a method of performing exercise counting false recognition filtering to improve exercise recognition performance for a specified exercise. According to an embodiment, the operations described in FIG. 14 may be performed heuristically, for example, in combination with the operations described in FIG. 3 to FIG. 13B, or may be heuristically performed as detailed operations of at least a part of the described operations.

In the electronic device (e.g., electronic device 101 in FIG. 1 and FIG. 2) according to an embodiment of the present disclosure, the method of filtering exercise counting false recognition to improve recognition performance of exercise counting may be performed, for example, according to the flowchart illustrated in FIG. 14. The flowchart illustrated in FIG. 14 is merely a flowchart according to an embodiment of improving the performance of exercise counting recognition for the electronic device 101, and the sequence of at least some operations may be modified, performed in parallel, performed as independent operations, or at least some additional operations may be performed complementarily to at least some of the existing operations. According to an embodiment of the present disclosure, operations 1401 to 1413 may be performed by at least one processor of the electronic device 101 (e.g., processor 120 in FIG. 1 and/or FIG. 2).

As illustrated in FIG. 14, the operation method performed by the electronic device 101 according to an embodiment (e.g., a method of filtering exercise counting false recognition) may include: extracting characteristic parameters on the basis of sensor data from a specified sensor (operation 1401), determining whether an amount of change in acceleration of the characteristic parameters satisfies a first condition of being between a first threshold and a second threshold (operation 1403), when the first condition is satisfied, determining whether an amount of change in angular velocity of the characteristic parameters satisfies a second condition of being between a third threshold and a fourth threshold (operation 1405), when the second condition is satisfied, determining whether an amount of change in atmospheric pressure of the characteristic parameters satisfies a third condition of being between a fifth threshold and a sixth threshold (operation 1407), when the third condition is satisfied, determining whether an acceleration peak-valley interval of the characteristic parameters satisfies a fourth condition of being between a seventh threshold and an eighth threshold (operation 1409), when all the first, second, third, and fourth conditions are satisfied, determining an exercise counting candidate section as an exercise motion (operation 1411), and when one of the first, second, third, or fourth conditions is not satisfied, determining the exercise counting candidate section as a non-exercise motion (operation 1413).

With reference to FIG. 14, at operation 1401, the processor 120 of the electronic device 101 may extract characteristic parameters on the basis of the sensor data from a specified sensor. According to an embodiment, the processor 120 may extract acceleration-based, gyro-based, and atmospheric pressure-based characteristic parameters in order to evaluate whether the exercise counting candidate section corresponds to an actual exercise motion or a non-exercise motion similar to an exercise motion. According to an embodiment, the characteristic parameters may include the amount of change in acceleration, acceleration peak-valley interval, amount of change in angular velocity, and amount of change in atmospheric pressure. According to an embodiment, the amount of change in each characteristic parameter may be calculated as a peak-to-peak (p2p) within the exercise counting candidate section.

At operation 1403, the processor 120 may determine whether the amount of change in acceleration of the characteristic parameters satisfies the specified first condition of being between the first threshold and the second threshold. According to an embodiment, when the amount of change in acceleration is not within a predetermined threshold range (e.g., between the first threshold and the second threshold), the processor 120 may filter the exercise counting candidate section as a non-exercise motion.

At operation 1403, when the amount of change in acceleration satisfies the specified first condition (e.g., “Yes” at operation 1403), then at operation 1405, the processor 120 may determine whether the amount of change in angular velocity of the characteristic parameters satisfies the specified second condition of being between the third threshold and the fourth threshold. According to an embodiment, when the amount of change in angular velocity is not within a predetermined threshold range (e.g., between the third threshold and the fourth threshold), the processor 120 may filter the exercise counting candidate section as a non-exercise motion.

At operation 1405, when the amount of change in angular velocity satisfies the specified second condition (e.g., “Yes” at operation 1405), then at operation 1407, the processor 120 may determine whether the amount of change in atmospheric pressure of the characteristic parameters satisfies the specified third condition of being between the fifth threshold and the sixth threshold. According to an embodiment, when the amount of change in atmospheric pressure is not within a predetermined threshold range (e.g., between the fifth threshold and the sixth threshold), the processor 120 may filter the exercise counting candidate section as a non-exercise motion.

At operation 1407, when the amount of change in atmospheric pressure satisfies the specified third condition (e.g., “Yes” at operation 1407), then at operation 1409, the processor 120 may determine whether the acceleration peak-valley interval of the characteristic parameters satisfies the specified fourth condition of being between the seventh threshold and the eighth threshold. According to an embodiment, when the acceleration peak-valley interval is not within a predetermined threshold range (e.g., between the seventh threshold and the eighth threshold), the processor 120 may filter the exercise counting candidate section as a non-exercise motion.

At operation 1409, when the acceleration peak-valley interval satisfies the specified fourth condition (e.g., “Yes” at operation 1409), then at operation 1411, the processor 120 may determine the exercise counting candidate section as an exercise motion.

At operation 1403, operation 1405, operation 1407, or operation 1409, when the characteristic parameter (e.g., amount of change in acceleration, amount of change in angular velocity, amount of change in atmospheric pressure, or acceleration peak-valley interval) does not satisfy one of the specified first condition, specified second condition, specified third condition, or specified fourth condition (e.g., “No” at operation 1403, “No” at operation 1405, “No” at operation 1407, or “No” at operation 1409), then at operation 1413, the processor 120 may determine the exercise counting candidate section as a non-exercise motion.

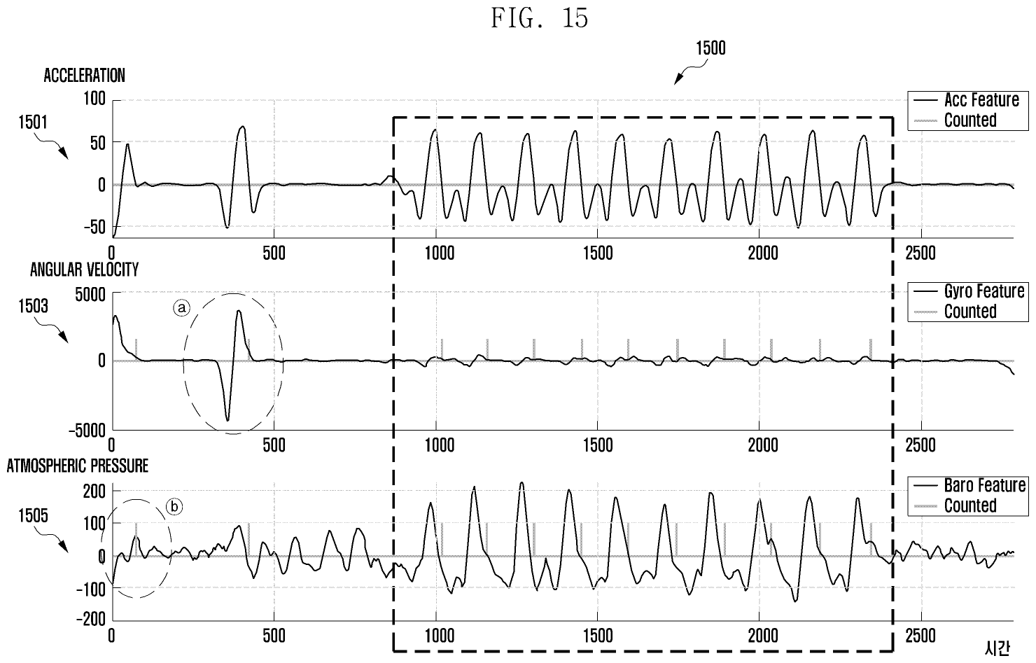

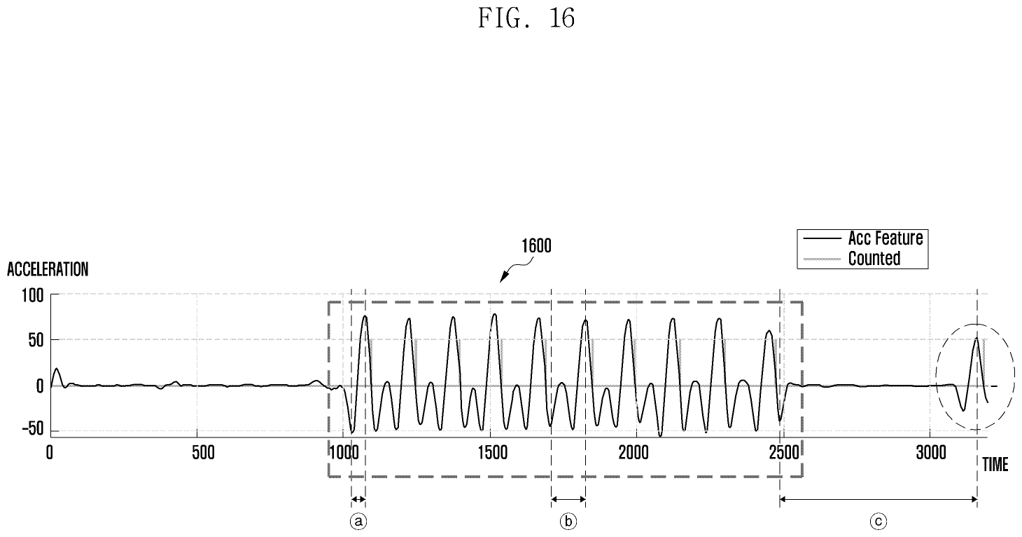

As illustrated in FIG. 14, an example of determining an actual exercise section on the basis of characteristic parameters is described with reference to the examples illustrated in FIG. 15 and FIG. 16.

FIG. 15 is a reference view for describing a false recognition filtering operation of exercise counting according to an embodiment of the present disclosure.

With reference to FIG. 15, the graph illustrated in FIG. 15 may, for example, represent an example of characteristic signals 1501, 1503, and 1505 from an acceleration sensor (e.g., acceleration sensor 240 in FIG. 2), a gyro sensor (e.g., gyro sensor 250 in FIG. 2), and an atmospheric pressure sensor (e.g., atmospheric pressure sensor 260 in FIG. 2), when the user performs a squat exercise ten repetitions with the hands clasped. For example, reference numeral 1501 may represent an example of a characteristic signal from the acceleration sensor 240, reference numeral 1503 may represent an example of a characteristic signal from the gyro sensor 250, and reference numeral 1505 may represent an example of a characteristic signal from the atmospheric pressure sensor 260. According to an embodiment, in FIG. 15, the x-axis in reference numerals 1501, 1503, and 1505 may represent time in seconds. According to an embodiment, in FIG. 15, the y-axis in reference numerals 1501, 1503, and 1505 may represent, respectively, acceleration in meters per second squared (m/s²), angular velocity in degrees per second (dps), and atmospheric pressure in hectopascals (hPa).

As illustrated in FIG. 15, in the characteristic signals 1501, 1503, and 1505 from the acceleration sensor 240, gyro sensor 250, and atmospheric pressure sensor 260, a portion 1500 marked by the dashed line may represent a section corresponding to an actual exercise motion, while the other sections outside of the portion 1500 marked by the dashed line may represent sections corresponding to non-exercise motions that is not the actual exercise motion. In an embodiment of the present disclosure, false recognition in exercise counting may be filtered by checking the amount of change in acceleration, the amount of change in angular velocity, and the amount of change in atmospheric pressure. In an embodiment, the amount of change in the characteristic signals from each sensor within the exercise counting candidate section may be calculated as a peak-to-peak (p2p).

According to an embodiment, in the graph of FIG. 15, in case of \textcircled{a} within the characteristic signal of the gyro sensor 250 of reference numeral 1503, it may represent a change amount much larger than that of the actual gyro change during exercise, and thus may not be counted as the exercise. According to an embodiment, in the graph of FIG. 15, in case of \textcircled{b} within the characteristic signal of the atmospheric pressure sensor 260 of reference numeral 1505, it may represent a change amount significantly smaller than that of the actual atmospheric pressure change during exercise, and thus may not be counted as the exercise.

FIG. 16 is a reference view for describing a false recognition filtering operation of exercise counting according to an embodiment of the present disclosure.

With reference to FIG. 16, the graph illustrated in FIG. 16 may, for example, represent an example of the characteristic signal from an acceleration sensor (e.g., acceleration sensor 240 in FIG. 2), when the user performs a squat exercise ten repetitions while holding a kettle bell between the legs. According to an embodiment, in FIG. 11, the x-axis may represent time (seconds), while the y-axis may represent acceleration (m/s²).

As illustrated in FIG. 16, the case in FIG. 16 may represent a case where, after the user has completed 10 repetitions of the exercise, a motion occurs in which the user raises the arm to view the screen of an electronic device (e.g., electronic device 101 in FIG. 1 and/or FIG. 2). For example, while both the upper boundary and lower boundary conditions are satisfied, a single exercise counting candidate section may be detected, and in this case, the amount of change in acceleration also satisfies the threshold range conditions, allowing the exercise counting candidate section to be counted as one repetition of the exercise.

In an embodiment of the present disclosure, in cases such as the above, false recognition in exercise counting may be filtered by checking the peak-valley interval between peaks and valleys within the exercise counting candidate section (e.g., the interval of an exercise counting candidate section that satisfies the conditions of the upper boundary and lower boundary). For example, as illustrated in the graph of FIG. 16, reference numeral 1600 may represent an actual exercise section. According to an embodiment, the sections \textcircled{a}, \textcircled{b}, and \textcircled{c} illustrated within the actual exercise section 1600 may represent exercise counting candidate sections that sequentially pass through the upper boundary and lower boundary. According to an embodiment, for exercise counting candidate sections such as sections \textcircled{a}, \textcircled{b}, and \textcircled{c} that satisfy the conditions of the upper boundary and lower boundary, the peak-valley interval between peaks and valleys may be checked. Therefore, sections within a specified predetermined threshold may be processed as exercise motions, while sections exceeding the specified predetermined threshold may be processed as non-exercise motions, thereby enhancing the accuracy of exercise counting. For example, as illustrated in the graph of FIG. 16, the peak-valley interval of section \textcircled{c} may be measured as relatively longer compared to the peak-valley intervals of sections \textcircled{a} and \textcircled{b}. For example, sections \textcircled{a} and \textcircled{b} may a section that satisfies the conditions of the upper boundary and lower boundary while being included within a predetermined threshold. For example, section \textcircled{c} may a section that satisfies the conditions of the upper boundary and lower boundary but that is equal to or greater than the predetermined threshold. In this manner, in case of \textcircled{c}, since the acceleration peak-valley interval falls outside the range of the predetermined threshold, the section \textcircled{c} may be determined as a non-exercise motion and thus may not be counted as the exercise.

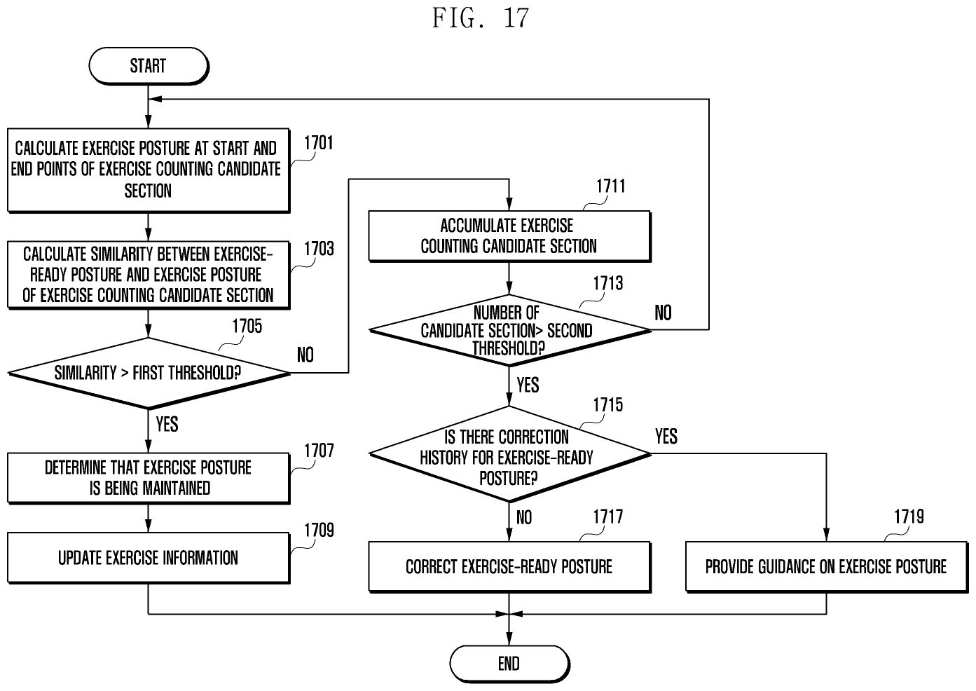

FIG. 17 is a flowchart illustrating a method of operating an electronic device according to an embodiment of the present disclosure.

According to an embodiment, FIG. 17 may illustrate an example of a method of determining whether an exercise posture is being maintained to improve the recognition performance of a specified exercise. According to an embodiment, the operations described in FIG. 17 may be performed heuristically, for example, in combination with the operations described in FIG. 3 to FIG. 16, or may be heuristically performed as detailed operations of at least a part of the described operations.

In an embodiment of the present disclosure, in the electronic device (e.g., electronic device 101 in FIG. 1 and/or FIG. 2), the method of determining whether an exercise posture is being maintained, to improve the recognition performance for exercise counting, may be performed, for example, according to the flowchart illustrated in FIG. 17. The flowchart illustrated in FIG. 17 is merely a flowchart according to an embodiment of improving the performance of exercise counting recognition for the electronic device 101, and the sequence of at least some operations may be modified, performed in parallel, performed as independent operations, or at least some additional operations may be performed complementarily to at least some of the existing operations. According to an embodiment of the present disclosure, operations 1701 to 1719 may be performed by at least one processor of the electronic device 101 (e.g., processor 120 in FIG. 1 and/or FIG. 2).

As illustrated in FIG. 17, an operation method (e.g., a method of determining whether an exercise posture is being maintained) performed by the electronic device 101 according to an embodiment may include: calculating an exercise posture at the start and end points of an exercise counting candidate section (operation 1701), calculating a similarity between an exercise-ready posture and an exercise posture of the exercise counting candidate section (operation 1703), determining whether the similarity exceeds a specified first threshold (operation 1705), when the similarity exceeds the specified first threshold, determining that the exercise posture is being maintained (operation 1707), updating exercise information (operation 1709), when the similarity does not exceed the specified first threshold (e.g., equal to or less than the specified first threshold), accumulating the exercise counting candidate section (operation 1711), determining whether the number of candidate sections exceeds a specified second threshold (operation 1713), when the number of candidate sections does not exceed the specified second threshold (e.g., equal to or less than the specified second threshold), returning to operation 1701, when the number of candidate sections exceeds the specified second threshold, determining whether there is a history of exercise-ready posture correction (operation 1715), when there is no history of exercise-ready posture correction, correcting the exercise-ready posture (operation 1717), and when there is a history of exercise-ready posture correction, providing guidance on the exercise posture (operation 1719).

With reference to FIG. 17, at operation 1701, the processor 120 of the electronic device 101 may calculate the exercise posture at the start and end points of the exercise counting candidate section.

At operation 1703, the processor 120 may calculate the similarity between the exercise-ready posture and the exercise posture of the exercise counting candidate section.

At operation 1705, the processor 120 may determine whether the similarity exceeds the specified first threshold.

At operation 1705, when the similarity exceeds the specified first threshold (e.g., “Yes” at operation 1705), then at operation 1707, the processor 120 may determine that the exercise posture is being maintained. According to an embodiment, the processor 120 may calculate the similarity between the initial exercise-ready posture and the posture within the exercise counting candidate section to determine whether the exercise posture is being maintained. According to an embodiment, when the similarity of the exercise posture exceeds a predetermined threshold, the processor 120 may determine that the user is maintaining the exercise posture properly while exercising. According to an embodiment, the similarity may be calculated using various similarity coefficients, such as cosine similarity and/or Pearson correlation coefficient.

At operation 1709, the processor 120 may update exercise information. According to an embodiment, the processor 120 may update exercise information (e.g., exercise repetitions, calories, and/or heart rate information).

At operation 1705, when the similarity does not exceed the specified first threshold (e.g., “No” at operation 1705), for example, when the similarity is equal to or less than the specified first threshold, then at operation 1711, the processor 120 may accumulate the exercise counting candidate section. According to an embodiment, the processor 120 may accumulate exercise counting candidate sections where the similarity of the exercise posture is equal to or less than the first threshold.

At operation 1713, the processor 120 may determine whether the number of candidate sections exceeds the specified second threshold.

At operation 1713, when the number of candidate sections does not exceed the specified second threshold (e.g., “No” at operation 1713), for example, when the number of candidate sections is equal to or less than the specified second threshold, the processor 120 may proceed to operation 1701 and perform the subsequent operations starting from operation 1701.

At operation 1713, when the number of candidate sections exceeds the specified second threshold (e.g., “Yes” at operation 1713), then at operation 1715, the processor 120 may determine whether there is a correction history for exercise-ready posture correction. According to an embodiment, when the number of accumulated exercise counting candidate sections exceeds a predetermined count (e.g., second threshold), the processor 120 may check whether there is a correction history for the exercise-ready posture.

At operation 1715, when there is no correction history for the exercise-ready posture (e.g., “No” at operation 1715), then at operation 1717, the processor 120 may correct the exercise-ready posture. According to an embodiment, when there is no correction history for the exercise-ready posture, the processor 120 may calculate a compensation value for the exercise-ready posture. For example, the compensation value for the exercise-ready posture may be applied to the initial exercise-ready posture, and the corrected exercise-ready posture may be used for determining whether the exercise posture is being maintained in subsequent evaluations.

At operation 1715, when there is a correction history for the exercise-ready posture (e.g., “Yes” at operation 1715), then at operation 1719, the processor 120 may provide guidance on the exercise posture. According to an embodiment, when there is a correction history for the exercise-ready posture, the processor 120 may provide guidance on the corresponding exercise posture to help the user assume the specified exercise posture.

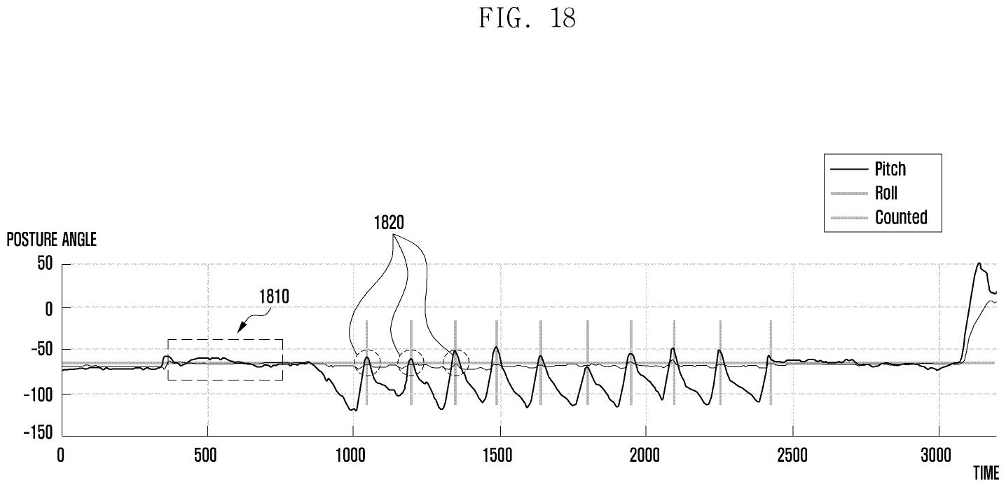

As illustrated in FIG. 17, an example of determining whether the exercise posture is being maintained is described with reference to the example illustrated in FIG. 18.

FIG. 18 is a reference view for describing the determination of whether an exercise posture is being maintained, according to an embodiment of the present disclosure.

With reference to FIG. 18, the graph illustrated in FIG. 18 may, for example, represent an example of exercise posture signals (e.g., roll and pitch) when the user performs a squat exercise 10 repetitions while holding a kettle bell between the legs. According to an embodiment, in FIG. 18, the x-axis represents time in seconds, while the y-axis may represent a posture angle (e.g., an angle in degrees of roll and pitch).

According to an embodiment, in conventional methods, the user's posture during exercise may be continuously monitored to determine whether the user has performed the motion of the specified exercise. As a result, when the user's exercise posture is disrupted even slightly during the exercise, there may be cases where the exercise is not counted.

In an embodiment of the present disclosure, the user's exercise posture (e.g., roll, pitch) may be checked in the initial exercise-ready posture recognition section, and subsequently, when the user exercises, only the start and end points of the exercise counting candidate section may be checked to determine whether the initial exercise-ready posture is being maintained. Therefore, even if the user's exercise posture is slightly disrupted during the exercise, the user's exercise may still be recognized and counted as the exercise.

For example, as illustrated in the graph of FIG. 18, the exercise posture in the initial exercise-ready posture recognition section (1810) before the start of the exercise may be calculated as approximately −65 degrees for both roll and pitch. Subsequently, it may be confirmed that the exercise posture at the start and end points 1820 of the exercise counting candidate section while the user is exercising maintains around approximately −65 degrees. In this manner, when the similarity between the posture in the initial exercise-ready posture recognition section 1810 and the posture in the exercise counting candidate section is equal to or greater than a predetermined threshold, the processor 120 may determine that the user is in a situation of maintaining the exercise posture.

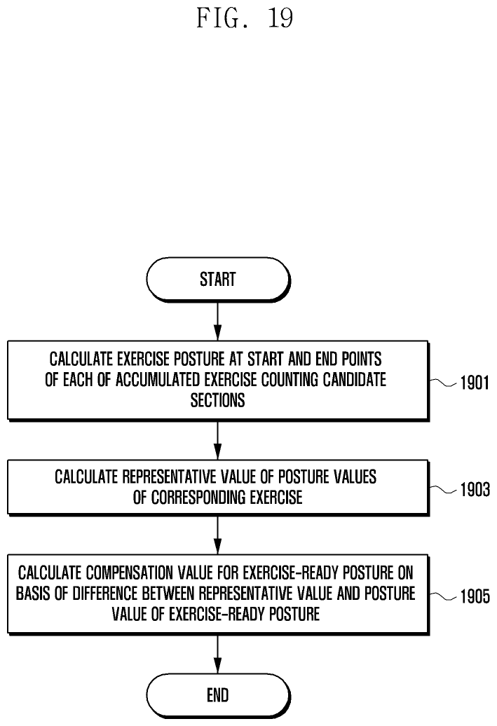

FIG. 19 is a flowchart illustrating a method of operating an electronic device according to an embodiment of the present disclosure.

According to an embodiment, FIG. 19 may illustrate an example of a method of correcting the exercise-ready posture to improve the recognition performance of a specified exercise. According to an embodiment, the operations described in FIG. 19 may be performed heuristically, for example, in combination with the operations described in FIG. 3 to FIG. 18, or may be heuristically performed as detailed operations of at least a part of the described operations.

In an embodiment of the present disclosure, in the electronic device (e.g., electronic device 101 in FIG. 1 and/or FIG. 2), the method of correcting the exercise-ready posture, to improve the recognition performance for exercise counting, may be performed, for example, according to the flowchart illustrated in FIG. 19. The flowchart illustrated in FIG. 19 is merely a flowchart according to an embodiment of improving the performance of exercise counting recognition for the electronic device 101, and the sequence of at least some operations may be modified, performed in parallel, performed as independent operations, or at least some additional operations may be performed complementarily to at least some of the existing operations. According to an embodiment of the present disclosure, operations 1901 to 1905 may be performed by at least one processor of the electronic device 101 (e.g., processor 120 in FIG. 1 and/or FIG. 2).

As illustrated in FIG. 19, an operation method (e.g., a method of correcting the exercise-ready posture) performed by the electronic device 101 according to an embodiment may include: calculating an exercise posture at the start and end points of each accumulated exercise counting candidate section (operation 1901), calculating an average value of corresponding exercise posture values (operation 1903), and calculating a compensation value for an exercise-ready posture on the basis of a difference between the average value and the posture value of the exercise-ready posture (operation 1905).

With reference to FIG. 19, at operation 1901, the processor 120 of the electronic device 101 may calculate the exercise posture at the start and end points of each accumulated exercise counting candidate section.

At operation 1903, the processor 120 may calculate a representative value for the corresponding exercise posture values. According to an embodiment, the processor 120 may calculate a representative value (e.g., maximum value, minimum value, median value, mode, or average value) of the exercise posture values that are calculated at the start and end points of each accumulated exercise counting candidate section.

At operation 1905, the processor 120 may calculate a compensation value for the exercise-ready posture on the basis of the difference between the representative value and the posture value of the exercise-ready posture. According to an embodiment, the compensation value for the exercise-ready posture may be applied to the initial exercise-ready posture, and the compensated exercise-ready posture may be used in determining whether the exercise posture is being maintained during subsequent exercises. According to an embodiment, for an section in which a compensation value for the exercise-ready posture has been calculated, the exercise count may be operated to be updated on the basis of the number of exercise counting candidate sections.

In an embodiment of the present disclosure, an operation method performed by the electronic device 101 may include: providing exercise guidance on the basis of detecting an exercise start trigger; recognizing the user's exercise-ready posture for a specified time period on the basis of sensor data from at least one specified sensor (e.g., acceleration sensor 240, gyro sensor 250, and/or atmospheric pressure sensor 260 in FIG. 2) of the sensor module (e.g., sensor module 176 or 230 in FIG. 1 or FIG. 2); providing exercise posture information related to the type of exercise corresponding to the exercise-ready posture; driving a recognition schema for the user's exercise counting corresponding to the exercise-ready posture; performing exercise counting based on the recognition schema; and providing exercise information according to the exercise counting.

According to an embodiment, the operation of recognizing the exercise-ready posture may include: recognizing the corresponding exercise posture based on the exercise-ready posture, comparing the exercise posture with a pre-specified reference exercise posture corresponding to the recognized exercise-ready posture, and determining whether the exercise posture is being maintained on the basis of a similarity between the exercise posture and the reference exercise posture.

According to an embodiment, the operation of driving the recognition schema may include: correcting the exercise-ready posture when the exercise posture does not substantially match the reference exercise posture, determining a first recognition schema as the recognition schema for counting the user's exercise according to the corrected exercise-ready posture, on the basis of the exercise-ready posture having been corrected, and determining a second recognition schema, different from the first recognition schema, as the recognition schema for counting the user's exercise according to the exercise-ready posture when the exercise posture substantially matches the reference exercise posture.

According to an embodiment, the operation of determining whether the exercise posture is being maintained may include: detecting an exercise counting candidate section on the basis of at least one sensor data from at least one specified sensor, filtering exercise motion and non-exercise motion for the exercise counting candidate section, calculating the exercise posture at the start and end points of the exercise counting candidate section, and calculating a similarity between the exercise-ready posture and the exercise posture of the exercise counting candidate section.

In a non-transitory computer-readable recording medium storing computer-executable instructions that, when executed by a processor 120 of an electronic device 101 individually and/or collectively, cause the electronic device 101 to perform operations, the operations may include: based on detecting an exercise start trigger, providing exercise guidance, recognizing an exercise-ready posture of a user for a specified time period based on sensor data from at least one specified sensor of the electronic device, providing exercise posture information related to a type of an exercise corresponding to the exercise-ready posture, driving a recognition schema for counting the exercise corresponding to the exercise-ready posture, performing exercise counting based on the recognition schema, and providing exercise information based on the exercise counting.

The electronic device according to various embodiments may be one of various types of electronic devices. The electronic devices may include, for example, a portable communication device (e.g., a smartphone), a computer device, a portable multimedia device, a portable medical device, a camera, a wearable device, a home appliance, or the like. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), the element may be coupled with the other element directly (e.g., wiredly), wirelessly, or via a third element.

As used in connection with various embodiments of the disclosure, the term “module” may include a unit implemented in hardware, software, or firmware, or any combination thereof, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the “non-transitory” storage medium is a tangible device, and may not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

According to the electronic device, operation method, and recording medium in accordance with an embodiment of the present disclosure, the recognition performance of exercise counting can be enhanced across various exercise postures of the user. According to an embodiment of the present disclosure, exercise counting recognition is possible even with various exercise postures, and exercise counts can be recognized even when the user's posture is slightly disrupted during the exercise. According to an embodiment of the present disclosure, even if the user performs a squat exercise with a posture different from the initial exercise-ready posture, the exercise count can still be recognized.

In addition, various effects that can be directly or indirectly identified through the present document may be provided. The effects obtained by the present disclosure are not limited to the aforementioned effects, and other effects, which are not mentioned above, will be clearly understood by those skilled in the art from the following description.

The various embodiments disclosed in the present specification and drawings are provided as examples merely for easily explaining the technical contents and helping understand the present disclosure, but not intended to limit the scope of the technology disclosed in the present disclosure. Therefore, the scope of the present disclosure should be interpreted that all changes or modified forms derived based on the technical spirit of the present disclosure fall within the scope of the present disclosure in addition to the embodiments disclosed herein.

What is claimed is:

1. An electronic device comprising:
a display;
at least one sensor;
memory storing instructions; and
at least one processor operatively connected to the display, the at least one sensor, and the memory,
wherein the instructions, when executed by the at least one processor individually and/or collectively, cause the electronic device to:
based on detecting an exercise start trigger, provide exercise guidance,
recognize an exercise-ready posture of a user for a specified time period based on sensor data from at least one specified sensor of the at least one sensor,
provide exercise posture information related to a type of an exercise corresponding to the exercise-ready posture,
drive a recognition schema for counting the exercise corresponding to the exercise-ready posture,
perform exercise counting based on the recognition schema, and
provide exercise information based on the exercise counting.

2. The electronic device of claim 1, wherein the instructions, when executed by the at least one processor individually and/or collectively, cause the electronic device to recognize the exercise counting based on a countable signal from the at least one specified sensor.

3. The electronic device of claim 1, wherein the instructions, when executed by the at least one processor individually and/or collectively, cause the electronic device to:
recognize an exercise posture based on the recognized exercise-ready posture, and
provide, during the recognition of the exercise posture, exercise posture information related to the recognized exercise-ready posture by displaying at least one of visual images or text on the display.

4. The electronic device of claim 3, wherein the instructions, when executed by the at least one processor individually and/or collectively, cause the electronic device to:
compare the exercise posture with a reference exercise posture corresponding to the recognized exercise-ready posture, and
determine whether the exercise posture is being maintained a similarity between the exercise posture and the reference exercise posture.

5. The electronic device of claim 4, wherein the instructions, when executed by the at least one processor individually and/or collectively, cause the electronic device to:
correct the exercise-ready posture in a state in which the exercise posture does not correspond to the reference exercise posture,
determine a first recognition schema as the recognition schema for counting the exercise corresponding to the corrected exercise-ready posture, based on the exercise-ready posture having been corrected,
determine a second recognition schema, different from the first recognition schema, as the recognition schema for counting the exercise corresponding to the exercise-ready posture in a state in which the exercise posture corresponds to the reference exercise posture, and
continuously accumulate and provide the exercise information according to the counting of the exercise based on the first recognition schema or the second recognition schema.

6. The electronic device of claim 1, wherein the instructions, when executed by the at least one processor individually and/or collectively, cause the electronic device to:
determine whether the exercise-ready posture is maintained for a predetermined time period based on a result of recognizing the exercise-ready posture during the specified time period, and
drive the recognition schema corresponding to the exercise-ready posture in a state in which the exercise-ready posture is maintained for the predetermined time period.

7. The electronic device of claim 1, wherein the instructions, when executed by the at least one processor individually and/or collectively, cause the electronic device to:
detect an exercise counting candidate section based on the sensor data from the at least one specified sensor,
filter an exercise motion and a non-exercise motion for the exercise counting candidate section,
determine an exercise posture at a start point and an end point of the exercise counting candidate section, and
determine a similarity between the exercise-ready posture and the exercise posture of the exercise counting candidate section.

8. The electronic device of claim 7, wherein the instructions, when executed by the at least one processor individually and/or collectively, cause the electronic device to:
extract a countable signal through signal processing of the sensor data from the at least one specified sensor, and
detect the exercise counting candidate section based on the extracted countable signal.

9. The electronic device of claim 8, wherein the instructions, when executed by the at least one processor individually and/or collectively, cause the electronic device to:
detect zero crossing points for the extracted countable signal,
detect peaks and valleys within a zero crossing section,
determine whether the peaks and valleys satisfy a specified condition based on whether the peaks and valleys pass through a predefined upper boundary and lower boundary,
check a sequence in which the countable signal passes through the predefined upper boundary and lower boundary within the zero crossing section in a state in which the specified condition is satisfied, and
determine a zero crossing section of the countable signal that satisfies the specified condition as the exercise counting candidate section.

10. The electronic device of claim 7, wherein the instructions, when executed by the at least one processor individually and/or collectively, cause the electronic device to perform filtering of exercise counting false recognition based on characteristic parameters for distinguishing between the exercise motion and the non-exercise motion, and
wherein the characteristic parameters comprise at least one of an amount of change in acceleration, an acceleration peak-valley interval, an amount of change in angular velocity, or an amount of change in atmospheric pressure.

11. The electronic device of claim 7, wherein the instructions, when executed by the at least one processor individually and/or collectively, cause the electronic device to:
determine that the user is maintaining the exercise posture in a state in which the similarity exceeds a threshold, and
update the exercise information.

12. The electronic device of claim 7, wherein the instructions, when executed by the at least one processor individually and/or collectively, cause the electronic device to:
determine that the exercise posture is not being maintained in a state in which the similarity is equal to or less than a threshold, and
correct the exercise-ready posture.

13. The electronic device of claim 12, wherein the instructions, when executed by the at least one processor individually and/or collectively, cause the electronic device to:
accumulate the exercise counting candidate section in a state in which the similarity is equal to or less than the threshold,
determine whether there is a correction history for the exercise-ready posture in a state in which a number of candidate sections exceeds a predetermined count,
correct the exercise-ready posture in a state in which there is no correction history for the exercise-ready posture, and
provide guidance on the exercise posture in a state in which there is the correction history for the exercise-ready posture.

14. The electronic device of claim 1, further comprising communication circuitry,
wherein the instructions, when executed by the at least one processor individually and/or collectively, cause the electronic device to:
control the communication circuitry to establish wireless communication with an external device based on detecting the exercise start trigger,
transmit visual information related to exercise coaching to the external device, and
enable the visual information related to the exercise coaching to be displayed through at least one of the display of the electronic device or a display of the external device.

15. A method of operating an electronic device, the method comprising:
based on detecting an exercise start trigger, providing exercise guidance;
recognizing an exercise-ready posture of a user for a specified time period based on sensor data from at least one specified sensor of the electronic device;
providing exercise posture information related to a type of an exercise corresponding to the exercise-ready posture;
driving a recognition schema for counting the exercise corresponding to the exercise-ready posture;
performing exercise counting based on the recognition schema; and
providing exercise information based on the exercise counting.

16. The method of claim 15, further comprising:
recognizing an exercise posture based on the exercise-ready posture,
comparing the exercise posture with a reference exercise posture corresponding to the recognized exercise-ready posture, and
determining whether the exercise posture is being maintained a similarity between the exercise posture and the reference exercise posture.

17. The method of claim 16, further comprising:
correcting the exercise-ready posture in a state in which the exercise posture does not correspond to the reference exercise posture,
determining a first recognition schema as the recognition schema for counting the exercise corresponding to the corrected exercise-ready posture, based on the exercise-ready posture having been corrected,
determining a second recognition schema, different from the first recognition schema, as the recognition schema for counting the exercise corresponding to the exercise-ready posture in a state in which the exercise posture corresponds to the reference exercise posture, and
continuously accumulating and providing the exercise information according to the counting of the exercise based on the first recognition schema or the second recognition schema.

18. The method of claim 15, further comprising:
determining whether the exercise-ready posture is maintained for a predetermined time period based on a result of recognizing the exercise-ready posture during the specified time period, and
driving the recognition schema corresponding to the exercise-ready posture in a state in which the exercise-ready posture is maintained for the predetermined time period.

19. The method of claim 15, further comprising:
detecting an exercise counting candidate section based on the sensor data from the at least one specified sensor,
filtering an exercise motion and a non-exercise motion for the exercise counting candidate section,
determining an exercise posture at a start point and an end point of the exercise counting candidate section, and
determining a similarity between the exercise-ready posture and the exercise posture of the exercise counting candidate section.

20. A non-transitory computer-readable recording medium storing computer-executable instructions that, when executed by a processor of an electronic device individually and/or collectively, cause the electronic device to perform operations, the operations comprising:
based on detecting an exercise start trigger, providing exercise guidance;
recognizing an exercise-ready posture of a user for a specified time period based on sensor data from at least one specified sensor of the electronic device;
providing exercise posture information related to a type of an exercise corresponding to the exercise-ready posture;
driving a recognition schema for counting the exercise corresponding to the exercise-ready posture;
performing exercise counting based on the recognition schema; and
providing exercise information based on the exercise counting.
